(19) United States
(12) Patent Application Publication
(10) Pub. No.: US 2025/0101529 A1
(43) Pub. Date: Mar. 27, 2025

(54) MULTIMODAL DETECTION WITH EXTRACELLULAR VESICLES

(71) Applicant: Mercy Bioanalytics, Inc., Waltham, MA (US)

(72) Inventors: Toumy Guettouche, Waltham, MA (US); Dawn Mattoon, Waltham, MA (US)

(21) Appl. No.: 18/894,419

(22) Filed: Sep. 24, 2024

Related U.S. Application Data
(60) Provisional application No. 63/585,029, filed on Sep. 25, 2023.

(51) Int. Cl. C12Q 1/6886; G01N 33/574

(52) U.S. Cl. CPC C12Q 1/6886; G01N 33/57492; G01N 2800/7028

(57) ABSTRACT

The invention provides methods for diagnosing disease by detecting extracellular vesicle-derived markers in combination with other diagnostic and screening assays. The methods include, but are not limited to, the analysis of extracellular vesicle-derived biomarkers obtained from bodily fluid samples. The methods include, but are not limited to, diagnostic and screening assays, including medical imaging. The present disclosure in one aspect provides technologies for detection and/or screening of a plurality of cancers. In another aspect, technologies provided herein are useful for selecting and/or monitoring and/or evaluating efficacy of, a treatment administered to a subject determined to have or susceptible to cancer.

### TECHNICAL FIELD

The present disclosure relates to methods for diagnosing disease by detecting extracellular vesicle-derived markers in combination with other diagnostic and screening assays.

### BACKGROUND

Diagnostic and screening assays are effective tools for sensitive and specific detection of diseases such as cancer. Conventional screening assays include a broad range of tests that measure analytes in biological samples, such as blood, urine, and others. For example, conventional venous blood draws measure cholesterol, A1C, liver enzymes, and various metabolic markers. Other, more specific, screening tests focus on a particular disease modality (e.g., HIV). In addition, some conventional screening diagnostics are based on imaging. For example, lung cancer screening and colonoscopy have been used to varying degrees of success.

The benefits of screening include early detection and the ability to treat or palliate a condition prior to the onset of disease and/or to minimize the impact of the disease. Test results are typically characterized in terms of their sensitivity (the likelihood that a positive result is a true positive) and specificity (the likelihood that patients who test negative truly are negative). Conventional screening diagnostics have varying levels of specificity and sensitivity and are known to produce false positive and/or false negative results. The impact of inaccurate results is either that a disease goes undetected until such time that it is more difficult to treat or that a patient receives treatment they do not require. Both false negative and false positive results have a significant impact on the lives of patients, costs, and general confidence in the value of screening tests. There is, therefore, a continuing need to develop screening and diagnostic tests that reduce the rate of false positive and false negative results.

### SUMMARY

The present invention provides improved methods for diagnosis and screening of disease by detecting extracellular vesicle-derived markers in combination with other assays.

According to the invention, a first assay measures the co-occurrence of two or more biomarkers of disease (e.g., cell-surface proteins, nucleic acids, and the like) that are associated with extracellular vesicles released from tumor cells. A second assay measures indicia of the disease based on metrics that are known not to be associated with extracellular vesicles. In a preferred embodiment, the first assay measures biomarkers present on or in an extracellular vesicle and the second assay measures biomarkers not associated with an extracellular vesicle. The biomarkers are selected from nucleic acids, proteins (including cell-surface proteins, antibodies, antigens and others), carbohydrate, sugars, and the like. In some embodiments of the invention, the second assay is an imaging assay, such as a CT scan, X-ray, or other imaging technique.

Any tissue or body fluid sample is useful in practice of the invention. Exemplary samples include blood, urine, cerebrospinal fluid, lymph, and saliva. In certain embodiments, the biological sample is a liquid sample that has been subjected to, for example, size exclusion chromatography to isolate extracellular vesicles. In certain other embodiments, the sample material is immobilized on a solid substrate. For example, the solid substrate can be a bead, including magnetic beads, cellulose, silica, gold, or an organic polymer, among others.

The invention includes and provides methods for the separation, isolation, purification, or extraction of both nucleic acids and EVs from a sample and from each other. Specific techniques are provided for separating nucleic acids from EVs and particularly for separating cell-free DNA (cfDNA) from the halo of proteins surrounding an EV. The sample may be subjected to multiple parallel assays for multimodal detection. In one example, a blood plasma or serum sample (4 ml) is processed for analysis of extracellular vesicles and nucleic acids, both those associated with an extracellular vesicle and those not associated with the extracellular vesicle. Extracellular vesicles are extracted using, for example, an immunoaffinity column. The cfDNA is then extracted from the supernatant. The cfDNA is also pulled off the extracellular vesicles by, for example size exclusion chromatography. The extracellular vesicle fraction and the cfDNA fraction can be isolated simultaneously or in any order in the assay and may be co-purified.

Any suitable technique or combination of the techniques are useful to separate DNA (or RNA) from EVs and to ensure that essentially all available nucleic acid is isolated or extracted from a sample. Suitable techniques include for example (i) chemical approaches, such as manipulating sample charge or stringency, (ii) electrophoretic-related methods, such as polyacrylamide gel electrophoresis (PAGE) or epitachophoresis, (iii) physical separation methods, such as size exclusion chromatography (SEC) or high performance liquid chromatography (HPLC), (iv) molecular methods using, for example, DNA-binding proteins or adaptor ligation, (v) other methods such as displacing bound cfDNA, and (vi) combinations of any of the methods.

In certain embodiments, the separated and isolated DNA is sequenced. Any other analysis may be performed. For example, assays of the invention may include methylation analysis, mutation analysis, fragmentation analysis (fragmentomics), RNA (oncoRNA, microRNA) analysis, and others known in the art. Preferred embodiments include a step comprising separation of DNA from EVs.

One set of techniques that may be used to separate DNA from EVs includes the manipulation of simple chemistry. For example, simply subjecting the sample to very low or very high stringency washes. The chemical conditions of high or low stringency (high or low temperature, salt, and pH) may be used to promote separation of cfDNA from EVs. Other chemical manipulations include charge manipulation. DNA may attached to EVs through the mediating effects of positively-charged proteins, the surface of EVs and DNA both tending to bear net negative charge. The sample may be exposed to other features with a strong positive charge such as protein (e.g., fixed to a surface such as a derivatized glass or silica bead), charged beads, surfaces or electrodes, or other such material that will attract and hold DNA more strongly than the positively charged proteins enmeshed in the corona or halo of the EV.

Another set of techniques that may be used to separate DNA from EVs includes methods generally related to electrophoresis. In one example, a sample is subjected to polyacrylamide gel electrophoresis. Certain preferred embodiments of the invention use isotachophoresis or epitachophoresis. In isotachophoresis, a sample is introduced between a zone of fast leading electrolyte (LE) and a zone of slow terminating (or: trailing) electrolyte (TE). More preferred embodiments specifically use epitachophoresis (ETP).

Another set of techniques that may be used to separate DNA from EVs includes methods of physical separation. In some embodiments the DNA attached to the extracellular vesicles are physically separated from the extracellular vesicles by ultra-centrifugation. In some embodiments the DNA attached to the extracellular vesicles are physically separated from the extracellular vesicles by chromatography e.g. High Performance Liquid Chromatography (HPLC) and Size Exclusion Chromatography (SEC). In some embodiments, the DNA attached to the extracellular vesicles is physically separated from the extracellular vesicles through size-based filtration. In certain aspects, the filtration comprises a multi-stage filter.

Another set of techniques that may be used to separate DNA from EVs includes methods of molecular separation i.e. by using, for example, degenerate oligonucleotides, major groove binders, minor groove binders, DNA Binding Proteins (DBP), or Methyl Binding Proteins. For example, a degenerate oligonucleotide may be designed that specifically binds to the DNA from EVs. This degenerate oligonucleotide may be attached to a solid substrate. The sample may be washed over the solid substrate attached to the degenerate oligonucleotides, wherein the DNA would attach to the degenerate oligonucleotides, and become separated from the EVs. In another example, a major groove binder e.g. pluramycins, aflatoxins, azinomycins, leinamycin, neocarzinostatin, and ditercalinium, may be attached to a solid substrate. The sample may be washed over the solid substrate attached to the major groove binder, wherein the DNA would attach to the major groove binder e.g. pluramycins, aflatoxins, azinomycins, leinamycin, neocarzinostatin, and ditercalinium, and become separated from the EVs. In another example, a minor groove binder e.g. Capecitabine, is attached to a solid substrate. The sample is washed over the solid substrate attached to the minor groove binder, wherein the DNA would attach to the minor groove binder e.g. Capecitabine, and become separated from the EVs. In another example, a DNA Binding Protein, e.g. FK506 binding protein 25, is attached to a solid substrate. The sample is washed over the solid substrate attached to the DNA Binding Protein, wherein the DNA would attach to the DNA Binding Protein e.g. FK506 binding protein 25, and become separated from the EVs. In another example, a Methyl Binding Protein e.g. MECP2, may be attached to a solid substrate. The sample is washed over the solid substrate attached to the Methyl Binding Protein, wherein the DNA would attach to the Methyl Binding Protein e.g. MECP2, and become separated from the EVs.

In some embodiments, the DNA is molecularly separated from the EVs by treating the sample with Deoxyribonuclease (DNase). DNase I, an endonuclease that non-specifically digests both ssDNA and dsDNA by hydrolysing phosphodiester bonds, facilitates an efficient digestion of external EV-DNA while maintaining the structure and integrity of EVs.

Alternatively, the DNA is separated from the EVs by using a commercially available kit for circulating DNA extraction (EXO-DNAc-PS; HansaBioMed OU, Estonia). Briefly, bead-bound extracellular vesicles were lysed with a lysis buffer and digested with proteinase K to release the DNA from protein complexes. The sample was then supplemented with ethanol, loaded onto a silica membrane spin column and centrifuged. Following centrifugation, the flow-through was discarded. Washing steps are performed to get rid of contaminating solvents and plasma-derived inhibitors before elution. The eluted DNA is supplemented with binding buffer and ethanol and loaded onto a new silica membrane spin column one more time for further purification and concentration. Two more washing steps are applied before eluting the purified DNA.

Alternatively, the DNA may be separated from the EVs by displacement through competitive binding after the addition of uninteresting nucleic acids, wherein the nucleic acids are ligation incompatible. In this example, the sample is flooded with random nucleic acids e.g. random DNA, in excess of the concentration of the DNA bound to the EVs. The excess random nucleic acids, e.g. random DNA, further binds to the EVs and causes DNA bound to the EVs to disassociate due to competitive binding properties. Once the DNA bound to the EVs are disassociated, they would be subject to elution. The excess nucleic acids may have ligation incompatible short 3′ overhang configurations with no potential for base pairing and/or a 5′ lacking a phosphate group to prevent ligation. The DNA may be separated from the EV prior to, simultaneously with, or after the isolation, purification, or extraction of extracellular vesicles.

In certain embodiments, a first assay comprises isolation of extracellular vesicles using, for example, a capture reagent, such as antibody-functionalized beads. In that case, the isolated extracellular vesicles are incubated with a plurality of antibody-DNA conjugates having DNA comprising single-strand overhang, such that members of the plurality have a single-stranded overhang that is complementary to that of a second antibody-DNA conjugate in the plurality. Complementary antibody-DNA conjugates bound to the same extracellular vesicle are sufficiently close to hybridize and then be ligated. The ligation products are detected using qPCR and a positive signal resulting from the co-occurrence of predetermined markers is indicative of a positive screen.

In certain instances, the oligo-linked probes comprise a binding moiety directed to an extracellular vesicle-associated membrane-bound polypeptide, which may be optionally conjugated to a solid substrate. An exemplary oligo-linked probe for an extracellular vesicle-associated membrane-bound polypeptide may comprise a solid substrate (e.g., a bead) and a binding moiety (e.g., an antibody agent) directed to an extracellular vesicle-associated membrane-bound polypeptide.

The invention contemplates any molecular biology technique for the identification, characterization, analysis, and/or quantification of markers. Such techniques include, but are not limited to, oligonucleotide probes, PCR, antibody-binding assays, antibody-DNA conjugates, and the like. Preferred embodiments focus on identification, characterization, analysis and/or quantification of markers associated with extracellular vesicles, such as exosomes, microvesicles, and apoptopic bodies. Exemplary markers include surface proteins (including transmembrane proteins), nucleic acids, cytosolic proteins, mitochondrial material, and lipids.

In a preferred embodiment, the predetermined markers and disease-specific markers are markers for cancer, such as bladder cancer, brain cancer, breast cancer, cervical cancer, chronic lymphocytic leukemia, chronic myeloid leukemia, colorectal cancer, endometrial cancer, esophageal cancer, gastrointestinal cancer, Hodgkin lymphoma, kidney cancer, liver cancer, lung cancer, multiple myeloma, non-Hodgkin lymphoma, ovarian cancer, pancreatic cancer, prostate cancer, sarcomas, skin cancer, and stomach cancer.

The first assay is designed to detect the co-occurrence of two or more disease biomarkers on the extracellular vesicle by contacting the sample material with oligo-linked probes, each directed to a predetermined marker. In some embodiments in which a predetermined marker is or comprises a surface protein marker and/or an intravesicular protein marker, the co-occurrence of two or more predetermined markers may be detected using, for example, a proximity ligation assay. The proximity ligation assay may comprise contacting sample material comprising extracellular vesicles with a set of oligo-linked probes, each directed to a predetermined marker. The set of oligo-linked probes comprises at least two distinct probes so that a combination comprising the extracellular vesicles and the set of probes is generated. Generally, the two probes each comprise: (i) a binding moiety directed to a surface protein marker and/or an intravesicular protein marker; and (ii) an oligonucleotide domain coupled to the binding moiety, the oligonucleotide domain comprising a double-stranded portion and a single-stranded overhang portion extended from one end of the oligonucleotide domain. Such single-stranded overhang portions of the probes are characterized in that they can hybridize to each other when the probes are bound to the same extracellular vesicle. Such a combination comprising the extracellular vesicles and the set of probes is then maintained under conditions that permit binding of the set of probes to their respective targets on the extracellular vesicles such that the probes can bind to the same extracellular vesicle to form a double-stranded complex. The double-stranded complex is detected by contacting it with a nucleic acid ligase to generate a covalently contiguous ligation product; and detecting the covalently contiguous ligation product. The ligation product is detected by amplification or sequencing. In a certain embodiment, the amplification is PCR and may be digital PCR, qPCR and the like, in each case that detects the ligation product using fluorescent hydrolysis probes. The presence of a covalently contiguous ligation product is indicative of the presence of extracellular vesicles that are positive for a predetermined marker of a disease. While a proximity ligation assay described above may perform better, e.g., with higher specificity and/or sensitivity than other proximity ligation assays, a person skilled in the art reading the present disclosure will appreciate that other forms of proximity ligation assays that are known in the art may be used in addition to other means for detecting extracellular vesicle-related markers.

In certain embodiments, the predetermined marker to be detected is a nucleic acid. Detection is accomplished by any suitable method, for example PCR, qPCR, sequencing, probe hybridization, ligase chain reaction, multiplex PCR, and others. Nucleic acid can be extracted using known methods, such as suspension in an extraction buffer (e.g., phosphate-buffered saline or EDTA) followed by cell lysis (e.g., with proteinase K) and nucleic acid is extracted using commercially-available kits, such as the Qiagen DNeasy Blood and Tissue Kit (Qiagen, Valencia CA).

In a preferred embodiment, the second assay comprises sequencing circulating tumor DNA (ctDNA) to detect somatic mutations or viral sequences as positive markers of squamous cell carcinoma (SCC). In certain embodiments, the somatic mutations are identified by comparison to a cancer atlas or prior sequencing information from the subject or the viral sequences are identified by comparison to human papilloma virus (HPV) or Epstein-Barr virus (EBV) genomic reference information. In certain embodiments, the second assay is an enzyme-linked immunosorbent assay (ELISA). Other assays include epigenomic assays (e.g., methylation), fragmentomics, metabolomics, structural variants, copy number alterations, insertions, deletions, rearrangements, and miRNAs.

In certain embodiments, the second assay is medical imaging. In some embodiments, the medical imaging is X-ray imaging or computed tomography (CT) imaging.

In a preferred embodiment, detection of the co-occurrence of the two or more predetermined markers is a positive test for lung adenocarcinoma, wherein the markers include at least two polypeptides, each expressed from a gene independently selected from the list consisting of CEACAM6, HS6ST2, PODXL2, LARGE2, MARCKSL1, MAL2, SMPDL3B, LSR, RAP2B, AP1M2, APOO, ALDH18A1, CDH3, NUP210, GPR160, RCC2, and LAMC2.

In a preferred embodiment, detection of the co-occurrence of the two or more predetermined markers is a positive test for cervical adenocarcinoma, wherein the markers include at least two polypeptides, each expressed from a gene independently selected from the list consisting of ILDR1, PODXL2, ULBP2, HS6ST2, LAMB3, LMNB1, AP1M2, CDH1, LAMC2, RAP2B, RACGAP1, LSR, CDH3, and EPCAM.

The invention contemplates methods for disease diagnosis generally, but is especially applicable to diagnosis and screening of cancer. The methods of the invention can be focused, e.g., on a particular type of cancer, or can be general in nature (e.g., multiplexed analysis of a plurality of conditions).

The invention also contemplates methods for analysis of disease progression, including severity, staging, and recurrence (including minimal residual disease). Methods of the invention track markers in extracellular vesicles for changes indicative of, for example, disease severity or stage. It is known, for example, that changes (increasing or decreasing amounts, structural alterations, sequence changes) in certain markers are associated with specific diseases. For example, exosome-based EFGR T790M has been associated with lung cancer and it has been noted that melanoma cells secrete increased amounts of exosome-associated PD-L1.

In a particular embodiment, methods of the invention are utilized to analyze cancer-related protein and glycosylation epitopes (PGEs) co-occurring on the surface of an extracellular vesicle. Preferred methods comprise detection of multiple cancer-related PGEs that co-occur on the same extracellular vesicle using immunoaffinity capture and proximity ligation followed by qPCR. In other instances, measurements are made inside the extracellular vesicle.

In some embodiments, the present disclosure identifies the source of a problem with certain prior technologies including, for example, certain conventional approaches to detection and diagnosis of lung cancer. For example, the present disclosure appreciates that many conventional diagnostic assays, e.g., X-ray imaging, sputum testing, low-dose CT scanning, and/or molecular tests based on cell-free nucleic acids, serum markers (e.g., carcinoembryonic antigen (CEA), cytokeratin 19 fragment (CYFRA 21-1), neuron-specific enolase (NSE), progastrin-releasing peptide (ProGRP), and/or squamous cell carcinoma antigen (SCCA)), and/or bulk analysis of extracellular vesicles, can be time-consuming, costly, and/or lacking sensitivity and/or specificity sufficient to provide a reliable and comprehensive diagnostic assessment.

The present disclosure provides technologies (including systems, compositions, and methods) that solve such problems, among other things, by detecting co-occurrence of markers that are a signature of specific diseases, such as cancer, in extracellular vesicles, which comprise extracellular vesicle-associated surface markers (including, e.g., membrane-bound and/or membrane-associated polypeptides) or a marker selected from the group consisting of internal markers, and RNA markers.

The invention takes advantage of the novel insight that there is a need for the development of an accurate method of detecting diseases such as cancer through the combination of detecting extracellular vesicles and markers associated therewith, with other screening methods, e.g., imaging methods such as, e.g., MRI, CT scan, etc.

The skilled artisan will appreciate additional aspects and advantages of the invention upon consideration of this disclosure and the detailed description and claims that follow.

### BRIEF DESCRIPTION OF THE DRAWINGS

FIG. 1. Shows an exemplary assay for detection of disease biomarkers on extracellular vesicles

FIG. 2. Shows workflow for a combination assay according to the invention.

FIG. 3. Shows an alternative combination assay according to the invention.

### DETAILED DESCRIPTION

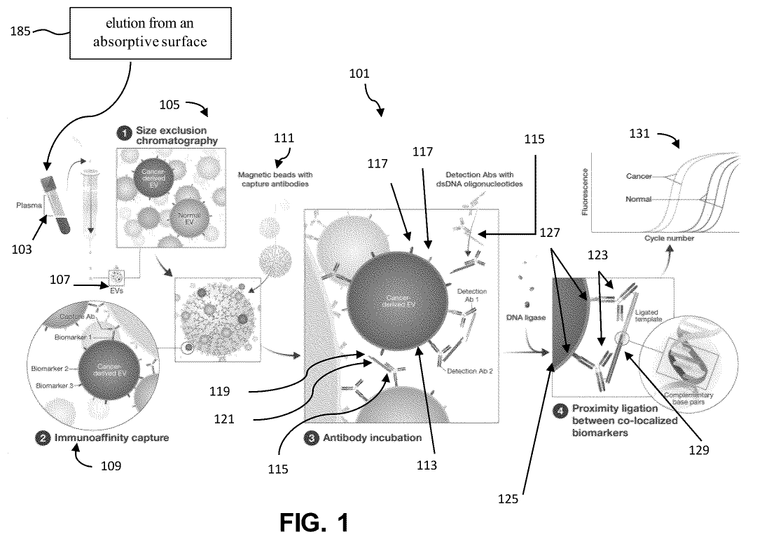

FIG. 1 shows an exemplary assay 101. As shown in the Figure, plasma 103 is obtained (e.g., from elution from an absorptive surface 185) and size exclusion chromatography 105 is used to isolate extracellular vesicles 107. FIG. 1 shows immunoaffinity capture 109 of extracellular vesicles 107 (exemplifying capture using antibody-functionalized beads 111). Captured extracellular vesicles 113 are incubated with antibody-DNA conjugates 115 targeting two surface markers 117. FIG. 1 shows a single-stranded overhang 119 on the double-stranded DNA portion 121 of the conjugate 115. As shown in FIG. 1, complementary antibody-DNA conjugates 123 bound to the same extracellular vesicle 125 are close enough to hybridize and become ligated (shown as proximity ligation between co-occurring markers 127). Finally, the ligation products 129 are analyzed using qPCR 131.

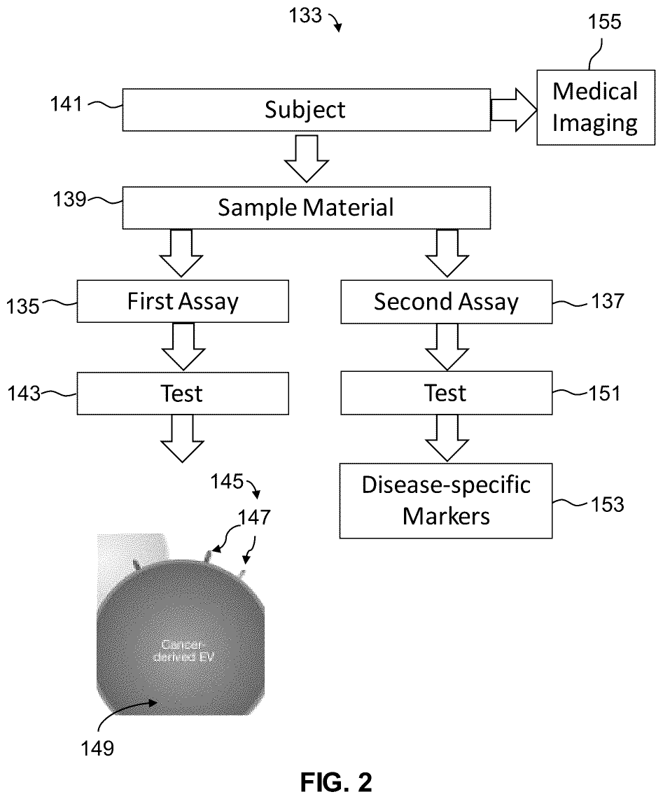

FIG. 2 diagrams a method 133 comprising performing a first assay 135 and a second assay 137 on sample material 139 from a subject 141, the first assay 135 comprising a test 143 for co-occurrence 145 of two or more predetermined markers 147 on an extracellular vesicle 149 isolated from the sample material 139 and the second assay 137 comprising a test 151 for disease-specific markers 153 that are not the markers 147 on the extracellular vesicle 149. FIG. 2 also shows an embodiment comprising medical imaging 155.

Any suitable assay may be used for the second assay 137. Suitable assays may be, but are not limited to, e.g., (i) physicals, general practitioner visits, cholesterol/lipid blood tests, diabetes (type 2) screening, colonoscopies, blood pressure screening, thyroid function tests, prostate cancer screening, mammograms, HPV/Pap smears, and/or vaccinations; (ii) chest X-ray imaging, sputum testing, chest low-dose CT scanning, and/or molecular tests based on cell-free nucleic acids, serum proteins (e.g., CEA, CYFRA 21-1, NSE, ProGRP, and/or SCCA); (iii) a genetic assay to screen blood plasma for genetic mutations in circulating tumor DNA and/or protein markers linked to cancer; (iv) an assay involving immunofluorescence staining to identify cell phenotype and marker expression, followed by amplification and analysis by next-generation sequencing; and/or (v) EGFR, KRAS, ALK, ROAS1, HER2, BRAF, and RET germline, somatic, and circulating cell mutation assays, or assays involving cell-free tumor DNA, liquid biopsy, serum protein and cell-free DNA, and/or circulating tumor cells.

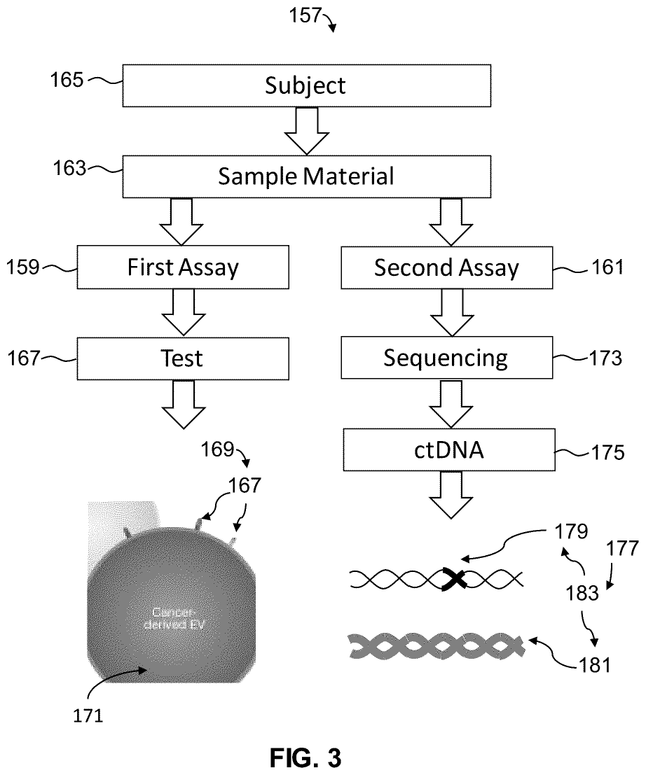

FIG. 3 diagrams a method 157 comprising performing a first assay 159 and a second assay 161 on sample material 163 from a subject 165, the first assay 159 comprising a test 167 for co-occurrence 169 of two or more predetermined markers for Adenocarcinoma 167 on an extracellular vesicle 171, and the second assay 161 comprising sequencing 173 circulating tumor DNA (ctDNA) 175 to detect 177 somatic mutations 179 or viral sequences 181 as positive markers of squamous cell carcinoma (SCC) 183.

Extracellular vesicles (EVs) generally include lipid bilayer-bound particles that are naturally released from almost all types of cells but, unlike a cell, cannot replicate. EVs range in diameter from near the size of the smallest physically possible unilamellar liposome (around 20-30 nanometers) to as large as 10 microns or more, although the vast majority of EVs are smaller than 200 nm. EVs can be divided according to size and synthesis route into exosomes, microvesicles and apoptotic bodies. They carry a cargo of proteins, nucleic acids, lipids, metabolites, and even organelles from the parent cell. Most cells that have been studied to date are thought to release EVs, including some archaeal, bacterial, fungal, and plant cells that are surrounded by cell walls.

In some embodiments, sample material is obtained or derived from a biological source (e.g., a tissue or organism or cell culture) of interest. In some embodiments, a source of interest may be or comprise a cell or an organism, such as an animal or human. In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humor, vomit, and/or combinations or component(s) thereof. In some embodiments, a biological fluid may be or comprise an intracellular fluid, an extracellular fluid, an intravesicular fluid (blood plasma), an interstitial fluid, a lymphatic fluid, and/or a transcellular fluid. In some embodiments, a biological tissue or sample material may be obtained, for example, by aspirate, biopsy (e.g., fine needle or tissue biopsy), swab (e.g., oral, nasal, skin, or vaginal swab), scraping, surgery, washing or lavage (e.g., bronchoalveolar, ductal, nasal, ocular, oral, uterine, vaginal, or other washing or lavage). In some embodiments, a biological sample material is or comprises a bodily fluid sample material or a bodily fluid-derived sample material. Examples of a bodily fluid sample material or a bodily fluid-derived sample material include, but are not limited to an amniotic fluid, bile, blood, breast milk, bronchoalveolar lavage fluid (BAL), cerebrospinal fluid, dialysate, feces, saliva, semen, synovial fluid, tears, urine, etc. In some embodiments, a bodily fluid sample material or a bodily fluid-derived sample material that may be useful in accordance with the present disclosure is or comprises a blood-derived sample, a saliva-derived sample, a sputum-derived sample, or a pleural effusion-derived sample. In some embodiments, a biological sample material is or comprises a liquid biopsy. In some embodiments, a biological sample material is or comprises cells obtained from an individual.

In some embodiments, capillary whole blood is obtained via a finger stick and drops of blood are spotted on a filter paper. The blood is dried and eluted for detection of extracellular vesicles that present markers of disease internally or on their surface. In one aspect, extracellular vesicles are eluted from dried blood spots and analyzed using conventional techniques, such as PCR, antibody detection, colorimetric readout, cell sorting techniques, probe binding and others. In a preferred embodiment of the invention, nanoparticles are eluted from a dried blood spot on cellulose or other absorptive material. The extracellular vesicles are then assayed for the presence, absence, or change in one or more markers. The markers may be surface proteins. For example, exosome surface proteins are highly heterogeneous and are characteristic of their tissues of origin. Accordingly, they are good markers for disease diagnosis, progression, and recurrence; as well as therapeutic selection and efficacy. In a particular embodiment, the markers are B-cell surface antigens, such as CD19, CD22, CD20, CD23, CD24, CD37, CD40, HLA markers, and the common leukocyte antigen, CD45, among others. In other embodiments, the marker is a nucleic acid and is detected by PCR and/or direct probe binding. In preferred embodiments, the marker detection is quantitative (e.g., by qPCR).

As discussed, any suitable sample may be obtained. The sample may be blood, e.g., obtained by a blood draw and collected in a blood collection tube, such as the blood collection tube sold under the trademark VACUTAINER by BD (Franklin Lakes, NJ). The sample may be subjected to multiple parallel assays or multimodal detection. The sample may be processed or treated prior to analysis by any suitable technique or method. For example, the sample may be processed to specifically extract or isolate different targets from within the sample such as extracellular vesicles (EVs) and other material such as nucleic acids, proteins, sugars, cellular fragments, or other material.

One insight of the invention is that EVs may be associated with or surrounded by a halo or corona comprising an assortment of proteins, nucleic acids, and other materials. See Wolf, 2022, A functional corona around extracellular vesicles enhances angiogenesis, skin regeneration and immunomodulation, J Extracell Vesicles 11:e12207, incorporated by reference. That is, an EV will typically have at least some cell-free DNA that is effectively stuck to a surface of the EV. The cfDNA may be attached to the EV by electrostatic forces, steric entanglement, van-der Vaals forces, hydrogen bonding, or other specific binding or attachment. Nevertheless, an insight of the invention is that segments of cfDNA may have important clinical utility. For example, cfDNA may be shed by tumors and any molecule of cfDNA may harbor a tumor-specific mutation, or tumor biomarker. Accordingly, the invention includes and provides methods for the separation, isolation, purification, or extraction of both nucleic acids and EVs from a sample and from each other. Specific techniques are provided for separating nucleic acids from EVs and particularly for separating cfDNA from the halo of proteins surrounding an EV.

Any suitable technique or combination of the techniques may be used to separate DNA from EVs and to ensure that essentially all available DNA is isolated or extracted from a sample. Suitable techniques may include generally (i) chemical approaches such as manipulating sample charge or stringency, (ii) electrophoretic-related methods such as polyacrylamide gel electrophoresis (PAGE) or epitachophoresis, (iii) physical separation methods such as size exclusion chromatography (SEC) or high performance liquid chromatography (HPLC), (iv) molecular methods using, for example, DNA-binding proteins or adaptor ligation, (v) other methods such as displacing bound cfDNA with other uninteresting nucleic acid, and (vi) combinations of any of the methods.

In general, when the sample is blood, methods of the disclosure work with samples of approximately 4 to 10 mL of blood, as is commonly collected in blood collection tubes. Typical workflows involve centrifugation and capture of the supernatant plasma, which typically yields about 3 to 5 mL of plasma. DNA may be purified from the plasma using known techniques such as treatment with proteinase K, incubation at temperature, and treatment with phenol/chloroform or using commercially available kits such as the circulating nucleic acid kit sold under the name QIAamp by Qiagen (Hilden, DE). It is understood that purified cfDNA typically exhibits a characteristic pattern of fragment sizes wherein a histogram of cfDNA fragment sizes would have a first, predominant peak for fragments of about 120-150 bases in length with a second peak for about 240 bases and another at about 450 bases. That periodicity corresponds to units of chromatin packing and even those fragments of about 120 bases may harbor a tumor-specific mutation such as the breakpoint of a structural variant that is a driver (or passenger) of a tumor. If one such fragment of cfDNA is attached to the EV corona and thus would otherwise escape isolation and sequencing, clinical information about tumor presence or status would be lost. Accordingly, the invention uses techniques to separate DNA from EVs.

As mentioned, the separated and isolated DNA may sequenced to detect mutations. Any other analysis may be performed. For example, assays of the invention may include methylation analysis, mutation analysis, fragmentation analysis (fragmentomics), RNA (oncoRNA, microRNA) analysis, or others. Preferred embodiments include a step or technique to separate DNA from EVs.

One set of techniques that may be used to separate DNA from EVs includes the manipulation of simple chemistry. For example, simply subjecting the sample to very low or very high stringency washes. While stringency generally refers to conditions relevant to Watson-Crick base pairing, the chemical conditions of high or low stringency (high or low temperature, salt, and pH) may be used to promote separation of cfDNA from EVs. Other chemical manipulations include charge manipulation. DNA may attached to EVs through the mediating effects of positively-charged proteins, the surface of EVs and DNA both tending to bear net negative charge. The sample may be exposed to other features with a strong positive charge such as protein (e.g., fixed to a surface such as a derivatized glass or silica bead), charged beads, surfaces or electrodes, or other such material that will attract and hold DNA more strongly than the positively charged proteins enmeshed in the corona or halo of the EV.

Another set of techniques that may be used to separate DNA from EVs includes methods generally related to electrophoresis, i.e., using an applied electric field to create a motive force on charged particles, which force is differential based on size and/or mass and thereby separates different components for each other. In one example, a sample is subject to polyacrylamide gel electrophoresis. Noting the characteristic fragment sizes described above for cfDNA and the characteristically larger size of EVs, the EVs and cfDNA fragments are driven to different positions in lanes of gels after which each band can be excised and the components re-suspended in a suitable media (e.g., buffer or, simply water). Certain preferred embodiments of the invention use isotachophoresis or epitachophoresis. In isotachophoresis, a sample is introduced between a zone of fast leading electrolyte (LE) and a zone of slow terminating (or: trailing) electrolyte (TE). Application of an electric potential results in a low electrical field in the leading electrolyte and a high electrical field in the terminating electrolyte. Analyte ions situated in the TE zone will migrate faster than the surrounding TE co-ions, while analyte ions situated in the LE will migrate slower; the result is that analytes are focused at the LE/TE interface. This will pull cfDNA away from EVs. More preferred embodiments specifically use epitachophoresis (ETP).

ETP utilizes a circular architecture in which a sample is loaded into an outer ring and pipetted out as a liquid extract from a central well. Using ETP, all nucleic acid can be separated from cells, EVs, and other components in single instrument run, and ETP has shown to be effective over a wide range of nucleic acid sizes. See Datinska, 2021, Epitachophoresis is a novel versatile total nucleic acid extraction method, Scientific Reports 11:a22736, incorporated by reference. When a sample such as a blood or plasma sample is subject to one of the electrophoresis-based methods such as epitachophoresis, the charge of the DNA (the very source of the problem) is exploited to cleanly separate each fragment of cfDNA from the halo or corona of an EV.

Another set of techniques that may be used to separate DNA from EVs includes methods of physical separation. In some embodiments the DNA attached to the surface of the extracellular vesicles are physically separated from the surface of the extracellular vesicles by ultra-centrifugation. In some embodiments the DNA attached to the surface of the extracellular vesicles are physically separated from the surface of the extracellular vesicles by chromatography e.g. High Performance Liquid Chromatography (HPLC) and Size Exclusion Chromatography (SEC). In some embodiments, the DNA attached to the extracellular vesicles is physically separated from the extracellular vesicles through size-based filtration. In certain aspects, the filtration comprises a multi-stage filter.

Another set of techniques that may be used to separate DNA from EVs includes methods of molecular separation i.e. by using, for example, degenerate oligonucleotides, major groove binders, minor groove binders, DNA Binding Proteins (DBP), or Methyl Binding Proteins. For example, a degenerate oligonucleotide primer may be designed that specifically bind to the DNA from EVs. This degenerate oligonucleotide may be attached to a solid substrate. The sample may be washed over the solid substrate attached to the degenerate oligonucleotides, wherein the DNA would attach to the degenerate oligonucleotides, and become separated from the EVs. In another example, a major groove binder e.g. pluramycins, aflatoxins, azinomycins, leinamycin, neocarzinostatin, and ditercalinium, may be attached to a solid substrate. The sample may be washed over the solid substrate attached to the major groove binder, wherein the DNA would attach to the major groove binder e.g. pluramycins, aflatoxins, azinomycins, leinamycin, neocarzinostatin, and ditercalinium, and become separated from the EVs. In another example, a minor groove binder e.g. Capecitabine, may be attached to a solid substrate. The sample may be washed over the solid substrate attached to the minor groove binder, wherein the DNA would attach to the minor groove binder e.g. Capecitabine, and become separated from the EVs. In another example, a DNA Binding Protein e.g. FK506 binding protein 25, may be attached to a solid substrate. The sample may be washed over the solid substrate attached to the DNA Binding Protein, wherein the DNA would attach to the DNA Binding Protein e.g. FK506 binding protein 25, and become separated from the EVs. In another example, a Methyl Binding Protein e.g. MECP2, may be attached to a solid substrate. The sample may be washed over the solid substrate attached to the Methyl Binding Protein, wherein the DNA would attach to the Methyl Binding Protein e.g. MECP2, and become separated from the EVs.

In some embodiments, the DNA is molecularly separated from the EVs by treating the sample with Deoxyribonuclease (DNase). DNase I, an endonuclease that non-specifically digests both ssDNA and dsDNA by hydrolysing phosphodiester bonds, facilitates an efficient digestion of external EV-DNA while maintaining the structure and integrity of EVs.

Alternatively, the DNA may be separated from the EVs by using a commercially available kit for circulating DNA extraction (EXO-DNAc-PS; HansaBioMed OU, Estonia). Briefly, bead-bound extracellular vesicles were lysed with a lysis buffer and digested with proteinase K to release the DNA from protein complexes. The sample was then supplemented with ethanol, loaded onto a silica membrane spin column and centrifuged. Following centrifugation, the flow-through was discarded. Washing steps were performed to get rid of contaminating solvents and plasma-derived inhibitors before elution. The eluted DNA was supplemented with binding buffer and ethanol and loaded onto a new silica membrane spin column one more time for further purification and concentration. Two more washing steps were applied before eluting the purified DNA.

Alternatively, the DNA may be separated from the EVs by displacement through competitive binding after the addition of uninteresting nucleic acids, wherein the nucleic acids are ligation incompatible. The sample may be flooded with random nucleic acids e.g. random DNA, in excess of the concentration of the DNA bound to the EVs. The excess random nucleic acids e.g. random DNA would further bind to the EVs and cause DNA bound to the EVs to disassociate due to competitive binding properties. Once the DNA bound to the EVs are disassociated, they would be subject to elution. The excess nucleic acids may have incompatible short 3′ overhang configurations with no potential for base pairing and/or a 5′ lacking a phosphate group to prevent ligation.

The DNA may be separated from the EV prior to, simultaneously with, or after the isolation, purification, or extraction of extracellular vesicles

In preferred embodiments, the invention provides methods for analyzing disease-associated markers comprising collecting capillary whole blood on an absorptive surface. The absorptive surface is selected from cellulose, filter paper, a textile surface, paper, gauze and the like. In certain embodiments, the absorptive surface is coated with an anticoagulant, such as ethylene diamine tetra-acetic acid (EDTA), however such coating is not necessary for successful use of the invention. Blood that is spotted on the absorptive surface is allowed to dry prior to analysis. Methods for drying blood spots are known in the art. See, e.g., Gruner, et al., J. Vis. Exp. 2015; (97): 52619, incorporated herein by reference. Dry blood sample material is eluted from the absorptive material for analysis.

In some embodiments sample material is obtained from a subject at two or more time points. A first assay comprising a test for co-occurrence of two or more predetermined markers is performed to identify the presence of the two or more predetermined markers in at least one of the samples, wherein at least one of the markers is produced stochastically in the sample. A positive screen is determined by the co-occurrence of two or more predetermined markers in at least one of the samples.

According to an embodiment of the present invention, a marker may be produced sporadically in situ during a first phase of a disease and is produced consistently in situ during a subsequent part of the disease. The marker may also be produced or expressed in the sample inconsistently during the earliest stages of the disease.

The present disclosure, among other things, provides insights and technologies for achieving effective cancer screening, e.g., for early detection of cancer (e.g., in some embodiments characterized by carcinoma, sarcoma, mixed types, etc.). In some embodiments, the present disclosure provides technologies for early detection of cancer in subjects who may be experiencing one more symptoms associated with cancer. In some embodiments, the present disclosure provides technologies for early detection of cancer in subjects who are at hereditary risks for cancer. In some embodiments, the present disclosure provides technologies for early detection of cancer in subjects who may be at hereditary risk and/or experiencing one or more symptoms associated with cancer. In some embodiments, the present disclosure provides technologies for early detection of cancer in subjects who may have life-history risk factors. In some embodiments, the present disclosure provides technologies for screening individuals, e.g., individuals with certain risks (e.g., hereditary risk, life history associated risk, or average risk) for early stage cancer (e.g., in some embodiments characterized by carcinoma, sarcoma, mixed types, etc.)). In some embodiments, provided technologies are effective for detection of early stage cancer (e.g., in some embodiments characterized by carcinoma, sarcoma, melanoma, and mixed types). In some embodiments, provided technologies are effective when applied to populations comprising or consisting of individuals having one or more symptoms that may be associated with cancer. In some embodiments, provided technologies are effective even when applied to populations comprising or consisting of asymptomatic or symptomatic individuals (e.g., due to sufficiently high sensitivity and/or low rates of false positive and/or false negative results). In some embodiments, provided technologies are effective when applied to populations comprising or consisting of individuals (e.g., asymptomatic or symptomatic individuals) without hereditary risk, and/or life-history related risk of developing cancer. In some embodiments, provided technologies are effective when applied to populations comprising or consisting of individuals (e.g., asymptomatic or symptomatic individuals) with hereditary risk for developing cancer. In some embodiments, provided technologies are effective when applied to populations comprising or consisting of individuals susceptible to cancer (e.g., individuals with a known genetic, environmental, or experiential risk, etc.). In some embodiments, provided technologies may be or include one or more compositions (e.g., molecular complexes, systems, collections, combinations, kits, etc.) and/or methods (e.g., of making, using, assessing, etc.), as will be clear to one skilled in the art reading the disclosure provided herein.

In some embodiments, provided technologies achieve detection (e.g., early detection, e.g., in asymptomatic individual(s) and/or population(s)) of one or more features (e.g., incidence, progression, responsiveness to therapy, recurrence, etc.) of cancer, with sensitivity and/or specificity (e.g., rate of false positive and/or false negative results) appropriate to permit useful application of provided technologies to single-time and/or regular (e.g., periodic) assessment. In some embodiments, provided technologies are useful in conjunction with an individual's regular medical examinations, such as but not limited to: physicals, general practitioner visits, cholesterol/lipid blood tests, diabetes screening (e.g., diabetes (type 2) screening), colonoscopies, blood pressure screening, thyroid function tests, prostate cancer screening, mammograms, HPV/Pap smears, and/or vaccinations. In some embodiments, provided technologies are useful in conjunction with treatment regimen(s); in some embodiments, provided technologies may improve one or more characteristics (e.g., rate of success according to an accepted parameter) of such treatment regimen(s).

In some embodiments, the present disclosure, among other things, provides insights that screening of asymptotic individuals, e.g., regular screening prior to or otherwise in absence of developed symptom(s), can be beneficial, and even important for effective management (e.g., successful treatment) of cancer. In some embodiments, the present disclosure provides cancer screening systems that can be implemented to detect cancer, including early-stage cancer, in some embodiments in asymptomatic individuals (e.g., without hereditary, and/or life-history associated risks in cancer). In some embodiments, provided technologies are implemented to achieve regular screening of asymptomatic individuals (e.g., with or without hereditary risk(s) in cancer). In some embodiments, provided technologies are implemented to achieve regular screening of symptomatic individuals (e.g., with or without hereditary and/or life-history associated risk(s) in cancer). The present disclosure provides, for example, compositions (e.g., reagents, kits, components, etc.), and methods of providing and/or using them, including strategies that involve regular testing of one or more individuals (e.g., asymptomatic individuals). The present disclosure defines usefulness of such systems, and provides compositions and methods for implementing them.

In some aspects, provided are technologies for use in classifying a subject (e.g., an asymptomatic subject) as having or being susceptible to cancer (e.g., carcinoma, sarcoma, mixed types, etc.). In some embodiments, the present disclosure provides methods or assays for classifying a subject (e.g., an asymptomatic subject) as having or being susceptible to cancer (e.g., carcinoma, sarcoma, mixed types, etc.). In some embodiments, a provided method or assay comprises assaying sample material (e.g., from a blood-derived sample) from a subject for a plurality of distinct predetermined marker combinations to determine in the sample material (e.g., from a blood-derived sample) whether extracellular vesicles display at least a marker combination from the plurality (e.g., co-occurrence of at least two markers), wherein the plurality of marker combinations each independently comprises at least two markers, whose combined expression level has been determined to be associated with at least one type of cancer (including, e.g., at least two types of cancer).

In a preferred embodiment, the predetermined markers 119 are markers for cancer. In a preferred embodiment, the disease-specific markers 127 are markers for cancer. In some embodiments, markers may also be referred to as biomarkers. Markers may be proteins, including co-localized surface proteins, cytosolic proteins, antigens, and may include mutations of any of the foregoing. In addition, biomarkers can be nucleic acids, epigenomic factors (e.g., methylation and the like) and may include mutations, such as deletions, inversions, substitutions, rearrangements, and others. In some embodiments, an assay for one or more provided markers of one or more marker combinations for cancer (e.g., ones described herein) includes a plurality of oligo-linked probes each for a specific target (e.g., a provided marker of a marker combination). In some embodiments, such a system may comprise at least 2, at least 3, at least 4, at least 5, at least 6, at least 7, at least 8, at least 9, at least 10, at least 15, at least 20, at least 25, at least 30, at least 40, at least 50, or more oligo-linked probes each for a specific target (e.g., a provided marker of a marker combination). In some embodiments, such a system may comprise 2-50 oligo-linked probes each for a specific target (e.g., a provided marker of a marker combination). In some embodiments, such a system may comprise 2-30 oligo-linked probes each for a specific target (e.g., a provided marker of a marker combination). In some embodiments, such a system may comprise 2-25 oligo-linked probes each for a specific target (e.g., a provided marker of a marker combination). In some embodiments, such a system may comprise 5-30 oligo-linked probes each for a specific target (e.g., a provided marker of a marker combination). In some embodiments, such a system may comprise 5-25 oligo-linked probes each for a specific target (e.g., a provided marker of a marker combination). In some embodiments, at least two of such oligo-linked probes in a set may be directed to the same marker of a marker combination. In some embodiments, at least two of such oligo-linked probes in a set may be directed to the same epitope of the same marker of a marker combination. In some embodiments, at least two of such oligo-linked probes in a set may be directed to different epitopes of the same marker of a marker combination.

In some embodiments, a provided method or assay comprises comparing sample information (determined from a subject's sample material) indicative of co-occurrence of markers for each marker combination to reference information including a reference threshold level for each marker combination.

In some embodiments, a provide method or assay comprises classifying a subject from which a sample material (e.g., from a blood-derived sample) is obtained as having or being susceptible to cancer when the sample material (e.g., from a blood-derived sample) shows that the co-occurrence of at least one marker combination is at or above a classification cutoff referencing a reference threshold level for the respective marker combination and optionally a reference threshold level for each other marker combination.

In some embodiments, a plurality of distinct marker combinations to be assayed in a sample material (e.g., from a blood-derived sample) includes at least 2 distinct marker combinations, including, e.g., at least 3, at least 4, at least 5, at least 6, at least 7, at least 8, at least 9, at least 10, at least 15, at least 20, at least 25, at least 30, or more distinct marker combinations.

In some embodiments, at least one marker combination within a selected plurality of marker combinations is specific for a tissue or organ type. By way of example only, in some embodiments, at least one marker combination may be specific for lung tissue. In some embodiments, at least one marker combination may be specific for colorectal tissue. In some embodiments, at least one marker combination may be specific for prostate tissue. In some embodiments, at least one marker combination may be specific for pancreatic tissue. In some embodiments, at least one marker combination may be specific for liver tissue. In some embodiments, at least one marker combination may be specific for bile duct tissue. In some embodiments, at least one marker combination may be specific for breast tissue. In some embodiments, at least one marker combination may be specific for esophageal tissue.

In some embodiments, at least one marker combination within a selected plurality of marker combinations may be associated with at least one particular type of cancer, including, e.g., at least two types of cancer or more. For example, in some embodiments, at least one marker combination may be associated with lung cancer. In some embodiments, at least one marker combination may be associated with colorectal cancer. In some embodiments, at least one marker combination may be associated with prostate cancer. In some embodiments, at least one marker combination may be associated with pancreatic cancer. In some embodiments, at least one marker combination may be associated with liver cancer. In some embodiments, at least one marker combination may be associated with bile duct cancer. In some embodiments, at least one marker combination may be associated with breast cancer. In some embodiments, at least one marker combination may be associated with esophageal cancer.

In some embodiments, at least one marker combination within a selected plurality of marker combinations is specific for a cell origin. By way of example only, in some embodiments, at least one marker combination may be specific for epithelial cells. In some embodiments, at least one marker combination may be specific for mesodermal cells. In some embodiments, at least one marker combination may be specific for fibroblast cells. In some embodiments, at least one marker combination may be specific for squamous cells.

In some embodiments, a target marker in a marker combination of cancer is or comprises an intravesicular marker, which is determined to be specific for certain cancers. In some embodiments, an intravesicular marker described herein may comprise at least one post-translational modification.

In some embodiments, a marker combination comprises one or more intravesicular RNA (e.g., but not limited to, mRNA and noncoding RNA such as, e.g., orphan noncoding RNA, long noncoding RNA, piwi-interacting RNA, microRNA, circular RNA, etc.) markers that have been determined to be associated with certain cancers.

In some embodiments, a marker combination for cancer comprises at least one or more (e.g., 1, 2, 3, 4, 5, 6, 7, 8, or more) extracellular vesicle-associated surface markers (e.g., ones described herein) and at least one or more (e.g., 1, 2, 3, 4, 5, 6, 7, 8, or more) surface markers (e.g., ones described herein). In some embodiments, at least one extracellular vesicle-associated surface marker and at least one surface marker are the same.

In some embodiments, at least one extracellular vesicle-associated surface marker and at least one surface marker(s) of a marker combination for cancer are distinct. For example, in some embodiments, a marker combination for cancer comprises at least one extracellular vesicle-associated surface marker and at least one surface marker.

In some embodiments, a marker combination for cancer comprises at least one or more (e.g., 1, 2, 3, 4, 5, 6, 7, 8, or more) surface marker (e.g., ones described herein) present on the surface of extracellular vesicles, (e.g., in some embodiments, extracellular vesicles having a size within the range of about 30 nm to about 1000 nm) and at least one or more (e.g., 1, 2, 3, 4, 5, 6, 7, 8, or more) intravesicular markers (e.g., ones described herein). In some such embodiments, the surface marker(s) and the intravesicular marker(s) can be encoded by the same gene, while the former is present on the surface of the extracellular vesicles and the latter is contained within the extracellular vesicle (e.g. cargo). In some such embodiments, the surface marker(s) and the intravesicular marker(s) can be encoded by different genes.

In some embodiments, a marker combination for cancer comprises at least one or more (e.g., 1, 2, 3, 4, 5, 6, 7, 8, or more) extracellular vesicle-associated surface markers (e.g., ones described herein) and at least one or more (e.g., 1, 2, 3, 4, 5, 6, 7, 8, or more) intravesicular markers (e.g., ones described herein). In some such embodiments, the extracellular vesicle-associated surface marker(s) and the intravesicular marker(s) can be encoded by the same gene, while the former is expressed in the membrane of the extracellular vesicle and the latter is contained within the extracellular vesicle (e.g., cargo). In some such embodiments, the extracellular vesicle-associated surface marker(s) and the intravesicular marker(s) can be encoded by different genes.

In some embodiments, a marker combination for cancer comprises at least one or more (e.g., 1, 2, 3, 4, 5, 6, 7, 8, or more) surface markers (e.g., ones described herein) and at least one or more (e.g., 1, 2, 3, 4, 5, 6, 7, 8, or more) intravesicular RNA (e.g., mRNA) markers (e.g., ones described herein). In some such embodiments, the surface marker(s) and the intravesicular RNA (e.g., but not limited to mRNA and noncoding RNA such as, e.g., orphan noncoding RNA, long noncoding RNA, piwi-interacting RNA, microRNA, circular RNA, etc.) marker(s) can be encoded by the same gene. In some such embodiments, the surface marker(s) and the intravesicular RNA (e.g., but not limited to mRNA and noncoding RNA such as, e.g., orphan noncoding RNA, long noncoding RNA, piwi-interacting RNA, microRNA, circular RNA, etc.) marker(s) can be encoded by different genes.

In some embodiments, a marker combination for cancer comprises at least one or more (e.g., 1, 2, 3, 4, 5, 6, 7, 8, or more) extracellular vesicle-associated surface markers (e.g., ones described herein) and at least one or more (e.g., 1, 2, 3, 4, 5, 6, 7, 8, or more) intravesicular RNA (e.g., but not limited to mRNA and noncoding RNA such as, e.g., orphan noncoding RNA, long noncoding RNA, piwi-interacting RNA, microRNA, circular RNA, etc.) markers (e.g., ones described herein). In some such embodiments, the extracellular vesicle-associated surface marker(s) and the intravesicular RNA (e.g., but not limited to mRNA and noncoding RNA such as, e.g., orphan noncoding RNA, long noncoding RNA, piwi-interacting RNA, microRNA, circular RNA, etc.) marker(s) can be encoded by the same gene. In some such embodiments, the extracellular vesicle-associated surface marker(s) and the intravesicular RNA (e.g., but not limited to mRNA and noncoding RNA such as, e.g., orphan noncoding RNA, long noncoding RNA, piwi-interacting RNA, microRNA, circular RNA, etc.) marker(s) can be encoded by different genes.

In some embodiments, any one of provided markers can be detected and/or measured by protein and/or RNA (e.g., mRNA) expression levels in wild-type form.

In some embodiments, any one of provided markers can be detected and/or measured by protein and/or RNA (e.g., mRNA) expression levels in mutant form. Thus, in some embodiments, mutant-specific detection of provided markers (e.g., proteins and/or RNA such as, e.g., mRNAs) can be included.

As noted herein, in some embodiments, a marker is or comprises a particular form of one or more polypeptides or proteins (e.g., a pro-form, a truncated form, a modified form such as a glycosylated, phosphorylated, acetylated, methylated, ubiquitylated, lipidated form, etc.). In some embodiments, detection of such form detects a plurality (and, in some embodiments, substantially all) polypeptides present in that form (e.g., containing a particular modification such as, for example, a particular glycosylation, e.g., sialyl-Tn (sTn) glycosylation, e.g., a truncated O-glycan containing a sialic acid α-2,6 linked to GalNAc α-O-Ser/Thr).

Accordingly, in some embodiments, a surface marker can be or comprise a glycosylation moiety (e.g., an sTn antigen moiety, a Tn antigen moiety, or a T antigen moiety). Thompsen-nouvelle (Tn) antigen is an O-linked glycan that is thought to be associated with a broad array of tumors. Tn is a single alpha-linked GalNAc added to Ser or Thr as the first step of a major O-linked glycosylation pathway. A skilled artisan will understand that in certain embodiments, T antigen typically refers to an O-linked glycan with the structure Galβ1-3GalNAc-.

In some embodiments, a surface protein marker can be or comprise a tumor-associated post-translational modification. In some embodiments, such a post-translational modification can be or comprise tumor-specific glycosylation patterns such as mucins with glycans aberrantly truncated at the initial GalNAc (e.g., Tn), or combinations thereof. In some embodiments, a surface protein marker can be or comprise a tumor-specific proteoform of mucin resulting from altered splicing and/or translation (isoforms) or proteolysis (cancer specific protease activity resulting in aberrant cleavage products).

In some embodiments, a marker combination is useful for detecting a subtype of cancer, for example, based on cell types. In some embodiments, a marker combination may be useful for detecting carcinoma. In some embodiments, a marker combination may be useful for detecting sarcoma.

In some embodiments, a marker combination is useful in detecting a subtype of cancer, for example, based on hormone status.

In general, the present disclosure provides technologies according to which a marker combination is analyzed and/or assessed in a sample material from a bodily fluid-derived sample (e.g., but not limited to, a blood-derived sample) comprising extracellular vesicles from a subject in need thereof; in some embodiments, a diagnosis or therapeutic decision is made based on such analysis and/or assessment.

In some embodiments, methods of detecting a marker combination include methods for detecting one or more provided markers of a marker combination as proteins, glycans, or proteoglycans (including, e.g., but not limited to, a protein with a carbohydrate or glycan moiety). Exemplary protein-based methods of detecting one or more provided markers include, but are not limited to, proximity ligation assay, mass spectrometry (MS) and immunoassays, such as immunoprecipitation; western blot; ELISA; immunohistochemistry; immunocytochemistry; flow cytometry; and immuno-PCR. In some embodiments, an immunoassay can be a chemiluminescent immunoassay. In some embodiments, an immunoassay can be a high-throughput and/or automated immunoassay platform.

In some embodiments, methods of detecting one or more provided markers as proteins, glycans, or proteoglycans (including, e.g., but not limited to a protein with a carbohydrate or glycan moiety) in a sample comprise contacting a sample with one or more antibody agents directed to the provided markers of interest. In some embodiments, such methods also comprise contacting the sample with one or more detection labels. In some embodiments, antibody agents are labeled with one or more detection labels.

In some embodiments, detecting binding between a marker of interest and an antibody agent for the marker of interest includes determining absorbance values or emission values for one or more detection agents. For example, the absorbance values or emission values are indicative of amount and/or concentration of marker of interest expressed by extracellular vesicles (e.g., higher absorbance is indicative of higher level of marker of interest expressed by extracellular vesicles). In some embodiments, absorbance values or emission values for detection agents are above a threshold value. In some embodiments, absorbance values or emission values for detection agents is at least 1.3, at least 1.4, at least 1.5, at least 1.6, at least 1.7, at least 1.8, at least 1.9, at least 2.0, at least 2.5, at least 3.0, at least 3.5 fold or greater than a threshold value. In some embodiments, the threshold value is determined across a population of a control or reference group (e.g., non-cancer subjects).

In some embodiments, methods of detecting one or more provided markers include methods for detecting one or more provided markers as nucleic acids. Exemplary nucleic acid-based methods of detecting one or more provided markers include, but are not limited to, performing nucleic acid amplification methods, such as polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). In some embodiments, a nucleic acid-based method of detecting one or more provided markers includes detecting hybridization between one or more nucleic acid probes and one or more nucleotide sequences that encode a marker of interest. In some embodiments, the nucleic acid probes are each complementary to at least a portion of one of the one or more nucleotide sequences that encode the marker of interest. In some embodiments, the nucleotide sequences s that encode the marker of interest include DNA (e.g., cDNA). In some embodiments, the nucleotide sequences that encode the marker of interest include RNA. In some embodiments, the nucleotide sequences that encode the marker of interest may be or comprise mRNA. In some embodiments, the nucleotide sequences that encode the marker of interest may be or comprise microRNA. In some embodiments, the nucleotide sequences that encode the marker of interest may be or comprise noncoding RNA, which in some embodiments may be or comprise orphan noncoding RNA (oncRNA). In some embodiments, the nucleotide sequences that encode the marker of interest may be or comprise long noncoding RNA (lncRNA). In some embodiments, the nucleotide sequences that encode the marker of interest may be or comprise piwi-interacting RNA (piwiRNA). In some embodiments, the nucleotide sequences that encode the marker of interest may be or comprise circular RNA (circRNA). In some embodiments, the nucleotide sequences that encode the marker of interest may be or comprise small nucleolar RNA (snoRNA).

In some embodiments, methods of detecting one or more provided markers involve proximity-ligation-immuno quantitative polymerase chain reaction (pliq-PCR). Pliq-PCR can have certain advantages over other technologies to profile EVs. For example, pliq-PCR can have a sensitivity three orders of magnitude greater than other standard immunoassays, such as ELISAs (Darmanis et al., 2010; which is incorporated herein by reference for the purpose described herein). In some embodiments, a pliq-PCR reaction can be designed to have an ultra-low LOD, which enables to detect trace levels of tumor-derived EVs, for example, down to a thousand EVs per mL.

In some embodiments, methods for detecting one or more provided markers may involve other technologies for detecting EVs, including, e.g., Nanoplasmic Exosome (nPLEX) Sensor (Im et al., 2014; which is incorporated herein by reference for the purpose described herein) and the Integrated Magnetic-Electrochemical Exosome (iMEX) Sensor (Jeong et al., 2016; which is incorporated herein by reference for the purpose described herein), which have reported LODs of ˜10³ and ˜10⁴ EVs, respectively (Shao et al., 2018; which is incorporated herein by reference for the purpose described herein).

In some embodiments, methods for detecting one or more provided markers in extracellular vesicles can be based on bulk EV sample analysis.

In some embodiments, methods for detecting one or more provided markers in extracellular vesicles can be based on profiling individual EVs (e.g., single-EV profiling assays).

A skilled artisan reading the present disclosure will understand that the assays described herein for detecting or profiling individual EVs can be also used to detect marker combinations on the surface of extracellular vesicles (e.g., as described herein).

In some embodiments, extracellular vesicles in a sample material may be captured or immobilized on a solid substrate prior to detecting one or more provided markers in accordance with the present disclosure. In some embodiments, extracellular vesicles may be captured on a solid substrate surface by non-specific interaction, including, e.g., adsorption. In some embodiments, extracellular vesicles may be selectively captured on a solid substrate surface. For example, in some embodiments, a solid substrate surface may be coated with an agent that specifically binds to extracellular vesicles (e.g., an antibody agent specifically targeting such extracellular vesicles, e.g., associated with cancer). In some embodiments, a solid substrate surface may be coated with a member of an affinity binding pair and an entity of interest (e.g., extracellular vesicles) to be captured may be conjugated to a complementary member of the affinity binding pair. In some embodiments, an exemplary affinity binding pair includes, e.g., but is not limited to biotin and avidin-like molecules such as streptavidin. As will be understood by those of skilled in the art, other appropriate affinity binding pairs can also be used to facilitate capture of an entity of interest to a solid substrate surface. In some embodiments, an entity of interest may be captured on a solid substrate surface by application of a current, e.g., as described in Ibsen et al. ACS Nano., 11: 6641-6651 (2017) and Lewis et al. ACS Nano., 12: 3311-3320 (2018), both of which are incorporated herein by reference for the purpose described herein, and both of which describe use of an alternating current electrokinetic microarray chip device to isolate extracellular vesicles from an undiluted human blood or plasma sample.

A solid substrate may be provided in a form that is suitable for capturing extracellular vesicles and does not interfere with downstream handling, processing, and/or detection. For example, in some embodiments, a solid substrate may be or comprise a bead (e.g., a magnetic bead). In some embodiments, a solid substrate may be or comprise a surface. For example, in some embodiments, such a surface may be a capture surface of an assay chamber (including, e.g., a tube, a well, a microwell, a plate, a filter, a membrane, a matrix, etc.). Accordingly, in some embodiments, a method described herein comprises, prior to detecting provided markers in a sample, capturing or immobilizing extracellular vesicles on a solid substrate.

In some embodiments, a sample material from a sample may be processed, e.g., to remove undesirable entities such as cell debris or cells, prior to capturing extracellular vesicles on a solid substrate surface. For example, in some embodiments, such a sample material may be subjected to centrifugation, e.g., to remove cell debris, cells, and/or other particulates. Additionally or alternatively, in some embodiments, such a sample material may be subjected to size-exclusion-based purification or filtration. Various size-exclusion-based purification or filtration are known in the art and those skilled in the art will appreciate that in some cases, a sample may be subjected to a spin column purification based on specific molecular weight or particle size cutoff. Those skilled in the art will also appreciate that appropriate molecular weight or particle size cutoff for purification purposes can be selected, e.g., based on the size of extracellular vesicles. For example, in some embodiments, size-exclusion separation methods may be applied to sample materials comprising extracellular vesicles to isolate a fraction of extracellular vesicles of a certain size (e.g., greater than 30 nm and no more than 1000 nm, or greater than 70 nm and no more than 200 nm). Typically, extracellular vesicles may range from 30 nm to several micrometers in diameter. See, e.g., Chuo et al., “Imaging extracellular vesicles: current and emerging methods” Journal of Biomedical Sciences 25: 91 (2018) which is incorporated herein by reference for the purpose described herein, which provides information of sizes for different extracellular vesicle (EV) subtypes: migrasomes (0.5-3 μm), microvesicles (0.1-1 μm), oncosomes (1-10 μm), exomeres (<50 nm), small exosomes (60-80 nm), and large exosomes (90-120 nm). In some embodiments, specific EV subtype(s) may be isolated, for example, in some embodiments by one or more size-exclusion separation methods, for detection assay.

In some embodiments, extracellular vesicles in a sample material may be processed prior to detecting one or more provided markers of a marker combination for cancer. Different sample material processing and/or preparation can be performed, e.g., to stabilize targets (e.g., target markers) in extracellular vesicles to be detected, and/or to facilitate exposure of targets (e.g., intravesicular proteins and/or RNA such as mRNA) to a detection assay (e.g., as described herein), and/or to reduce non-specific binding. Examples of such sample material processing and/or preparation are known in the art and include, but are not limited to, crosslinking molecular targets (e.g., fixation), permeabilization of biological entities (e.g., cells or extracellular vesicles), and/or blocking non-specific binding sites.

In one aspect, the present disclosure provides a method for detecting whether a marker combination of cancer is present or absent in a biological sample from a subject in need thereof, which may be in some embodiments a biological sample material (e.g., but not limited to, from a blood-derived sample) comprising extracellular vesicles. In some embodiments, such a method comprises (a) detecting, in a biological sample material such as a bodily fluid sample material (e.g., in some embodiments, from a blood-derived sample such as, e.g., a plasma sample) from a subject, extracellular vesicles having a marker combination of cancer; and (b) comparing sample information indicative of the co-occurrence of the marker combination-expressing biological extracellular vesicles in the biological sample material (e.g., a bodily fluid sample material such as, e.g., in some embodiments, from a blood-derived sample) to reference information including a reference threshold level. In some embodiments, a reference threshold level corresponds to a level of extracellular vesicles that express such a marker combination in comparable samples from a population of reference subjects, e.g., non-cancer subjects. In some embodiments, exemplary non-cancer subjects include healthy subjects (e.g., healthy subjects of specified age ranges, such as e.g., below age 55 or above age 55), subjects with non-cancer-related health diseases, disorders, or conditions (including, e.g., subjects having benign tumors, inflammatory disorders, etc.), and combinations thereof.

In some embodiments, a sample material is pre-screened for certain characteristics prior to utilization in an assay as described herein. In some embodiments, a sample material meeting certain pre-screening criteria is more suitable for diagnostic applications than a sample failing pre-screening criteria. For example, in some embodiments, sample material is visually inspected for appearance using known standards, e.g., is the sample normal, hemolyzed (red), icteric (yellow), and/or lipemic (whitish/turbid). In some embodiments, sample material can then be rated on a known standard scale (e.g., 1, 2, 3, 4, 5) and the results are recorded. In some embodiments, sample material is visually inspected for hemolysis (e.g., heme) and rated on a scale from 1-5, where the visual inspection correlates with a known concentration, e.g., where 1 denotes approximately 0 mg/dL, 2 denotes approximately 50 mg/dL, 3 denotes approximately 150 mg/dL, 4 denotes approximately 250 mg/dL, and 5 denotes approximately 525 mg/dL. In some embodiments, samples are visually inspected icteric levels (e.g., bilirubin) and rated on a scale from 1-5, where the visual inspection correlates with a known concentration, e.g., where 1 denotes approximately 0 mg/dL, 2 denotes approximately 1.7 mg/dL, 3 denotes approximately 6.6 mg/dL, 4 denotes approximately 16 mg/dL, and 5 denotes approximately 30 mg/dL. In some embodiments, sample material is visually inspected for turbidity (e.g. lipids) and rated on a scale from 1-5, where the visual inspection correlates with a known concentration, e.g., where 1 denotes approximately 0 mg/dL, 2 denotes approximately 125 mg/dL, 3 denotes approximately 250 mg/dL, 4 denotes approximately 500 mg/dL, and 5 denotes approximately 1000 mg/dL.

In some embodiments, sample materials scoring lower than a certain level on one or more metrics, e.g., equal to or lower than a score of 4, may be utilized in an assay as described herein. In some embodiments, samples scoring lower than a certain level on one or more metrics, e.g., equal to or lower than a score of 3, may be utilized in an assay as described herein. In some embodiments, samples scoring lower than a certain level on one or more metrics, e.g., equal to or lower than a score of 2, may be utilized in an assay as described herein. In some embodiments, samples scoring lower than a certain level on all three metrics (e.g., hemolyzed, icteric, and lipemic) e.g., equal to or lower than a score of 2, may be utilized in an assay as described herein. In some embodiments, low visual inspection scores on pre-screening criteria such as hemolysis, bilirubin, and/or lipemia (e.g., equal to or lower than a score of 2) may have no appreciable effect (e.g., not be correlated with) on diagnostic properties (e.g., Ct values) produced in an assay as described herein.

In some embodiments, a sample material is determined to be positive for the presence of a marker combination (e.g., ones described herein) when it shows an elevated level of extracellular vesicles that present the marker combination on their surface, relative to a reference threshold level (e.g., ones described herein). In some embodiments, a sample material is determined to be positive for the presence of a marker combination (e.g., as reflected by the level of marker combination-expressing extracellular vesicles) if its level is at least 30% or higher, including, e.g., at least 40%, at least 50%, at least 60%, at least 70%, at least 80%, at least 90%, at least 95% or higher, as compared to a reference threshold level. In some embodiments, a sample is determined to be positive for the presence of a marker combination (e.g., as reflected by the level of marker combination-expressing extracellular vesicles) if its level is at least 2-fold or higher, including, e.g., at least 3-fold, at least 4-fold, at least 5-fold, at least 6-fold, at least 7-fold, at least 8-fold, at least 9-fold, at least 10-fold, at least 50-fold, at least 100-fold, at least 250-fold, at least 500-fold, at least 750-fold, at least 1000-fold, at least 2500-fold, at least 5000-fold, or higher, as compared to a reference threshold level.

In some embodiments, a binary classification system may be used to determine whether a sample material is positive for the presence of a marker combination (e.g., ones described herein). For example, in some embodiments, a sample material is determined to be positive for the presence of a target marker signature (e.g., as reflected by the level of marker combination-expressing extracellular vesicles) if its level is at or above a reference threshold level, e.g., a cutoff value. In some embodiments, such a reference threshold level (e.g., a cutoff value) may be determined by selecting a certain number of standard deviations away from an average value obtained from control subjects such that a desired sensitivity and/or specificity of a cancer detection assay (e.g., ones described herein) can be achieved. In some embodiments, such a reference threshold level (e.g., a cutoff value) may be determined by selecting a certain number of standard deviations away from a maximum assay signal obtained from control subjects such that a desired sensitivity and/or specificity of a cancer detection assay (e.g., ones described herein) can be achieved. In some embodiments, such a reference threshold level (e.g., a cutoff value) may be determined by selecting the less restrictive of either (i) a certain number of standard deviations away from an average value obtained from control subjects, or (ii) a certain number of standard deviations away from a maximum assay signal obtained from control subjects, such that a desired sensitivity and/or specificity of a cancer detection assay (e.g., ones described herein) can be achieved. In some embodiments, control subjects for determination of a reference threshold level (e.g., a cutoff value) may include, but are not limited to healthy subjects, subjects with inflammatory conditions (e.g., Crohn's disease, ulcerative colitis, endometriosis, etc.), subjects with benign tumors, and combinations thereof. In some embodiments, healthy subjects and subjects with inflammatory conditions that are associated with tissues of interest but that are not cancerous (including, e.g., atherosclerosis, heart disease, chronic kidney disease, diabetes, inflammatory bowel disease, fatty liver disease, chronic obstructive pulmonary disease, endometriosis, rheumatoid arthritis, obesity, pancreatitis etc.) are included in determination of a reference threshold level (e.g., a cutoff value). In some embodiments, subjects with benign tumors are not included in determination of a reference threshold level (e.g., a cutoff value). In some embodiments, a reference threshold level (e.g., a cutoff value) may be determined by selecting at least 1.5 standard deviations (SDs) or higher (including, e.g., at least 1.6, at least 1.7, at least 1.8, at least 1.9, at least 2, at least 2.1, at least 2.2, at least 2.3, at least 2.4, at least 2.5, at least 2.6, at least 2.7, at least 2.8, at least 2.9, at least 3, at least 3.1, at least 3.2, at least 3.3, at least 3.4, at least 3.5, at least 3.6 or higher SDs) away from (i) an average value obtained from control subjects, or (ii) a maximum assay signal obtained from control subjects, such that a desired specificity (e.g., at least 95% or higher specificity [including, e.g., at least 96%, at least 97%, at least 98%, at least 99%, or higher specificity] such as in some embodiments at least 99.8% specificity) of a cancer detection assay (e.g., ones described herein) can be achieved. In some embodiments, a reference threshold level (e.g., a cutoff value) may be determined by selecting at least 2.9 SDs (e.g., at least 2.93 SDs) away from (i) an average value obtained from control subjects, or (ii) a maximum assay signal obtained from control subjects, such that a desired specificity (e.g., at least 99%, or higher specificity) of a cancer detection assay (e.g., ones described herein) can be achieved. In some embodiments, a reference threshold level (e.g., a cutoff value) may be determined by selecting at least 2.9 SDs (e.g., at least 2.93 SDs) away from the less restrictive of (i) an average value obtained from control subjects, or (ii) a maximum assay signal obtained from control subjects, such that a desired specificity (e.g., at least 99%, or higher specificity) of a cancer detection assay (e.g., ones described herein) can be achieved. In some embodiments, such a reference threshold level (e.g., a cutoff value) may be determined based on expression level (e.g., transcript level) of a target marker in normal healthy tissues vs. in cancer samples such that the specificity and/or sensitivity of interest (e.g., as described herein) can be achieved. In some embodiments, a reference threshold level (e.g., a cutoff value) may vary dependent on, for example, cancer stages and/or subtypes and/or patient characteristics, for example, patient age, risks factors for cancer (e.g., hereditary risk vs. average risk, life-history-associated risk factors), symptomatic/asymptomatic status, and combinations thereof.

In some embodiments, a reference threshold level (e.g., a cutoff value) may be determined based on a log-normal distribution around healthy subjects (e.g., of specified age ranges), and optionally subjects with inflammatory conditions that are associated with tissues of interest but that are not cancerous (including, e.g., atherosclerosis, heart disease, chronic kidney disease, diabetes, inflammatory bowel disease, fatty liver disease, chronic obstructive pulmonary disease, endometriosis, rheumatoid arthritis, obesity, pancreatitis etc.), and selection of a level that is necessary to achieve the specificity of interest, e.g., based on prevalence of cancer or a subtype thereof (e.g., including but not limited to in some embodiments characterized by carcinoma, sarcoma, melanoma, and mixed types). In some embodiments, specificity of interest may be at least 70%, including, e.g., at least 75%, at least 80%, at least 85%, at least 90%, at least 95%, at least 98%, at least 99%, at least 99.5% or higher.

The present disclosure, among other things, also provides technologies for determining whether a subject as having or being susceptible to cancer, for example, from a sample material that includes extracellular vesicles. For example, in some embodiments, when a biological sample material (e.g., a bodily fluid sample material from, e.g., but not limited to, a blood-derived sample) from a subject in need thereof shows a level of marker combination-expressing extracellular vesicles that is at or above a reference threshold level, e.g., cutoff value (e.g., as determined in accordance with the present disclosure), then the subject is classified as having or being susceptible to cancer. In some such embodiments, a reference threshold level (e.g., cutoff value) may be determined based on a log-normal distribution around healthy subjects (e.g., of specified age ranges), and optionally subjects with inflammatory conditions that are associated with tissues of interest but that are not cancerous (including, e.g., atherosclerosis, heart disease, chronic kidney disease, diabetes, inflammatory bowel disease, fatty liver disease, chronic obstructive pulmonary disease, endometriosis, rheumatoid arthritis, obesity, pancreatitis etc.) and selection of a level that is necessary to achieve the specificity of interest, e.g., based on prevalence of cancer or a subtype thereof (e.g., in some embodiments characterized by carcinoma, sarcoma, melanoma, and mixed types). In some embodiments, specificity of interest may be at least 70%, including, e.g., at least 75%, at least 80%, at least 85%, at least 90%, at least 95%, at least 98%, at least 99%, at least 99.5% or higher.

In some embodiments, a reference threshold level (e.g., a cutoff value) may be determined based on expression level (e.g., transcript level) of individual target marker(s) of a marker combination in normal healthy tissues vs. in cancer samples such that the specificity and/or sensitivity of interest (e.g., as described herein) can be achieved. In some embodiments, a reference threshold level (e.g., a cutoff value) may vary dependent on, for example, cancer stages and/or subtypes and/or patient characteristics, for example, patient age, risks factors for cancer (e.g., hereditary risk vs. average risk, life-history-associated risk factors), symptomatic/asymptomatic status, and combinations thereof.

In some embodiments, when a biological sample material from a subject in need thereof shows a level of marker combination that satisfies a reference threshold level, then the subject is classified as having or being susceptible to cancer. For example, in some embodiments, when a biological sample material (e.g., a bodily fluid sample material from, e.g., but not limited to, a blood-derived sample) from a subject in need thereof shows an elevated level of marker combination-expressing extracellular vesicles relative to a reference threshold level, then the subject is classified as having or being susceptible to cancer. In some embodiments, a subject in need thereof is classified as having or being susceptible to cancer when the subject's biological sample (e.g., a bodily fluid sample such as, e.g., but not limited to a blood-derived sample) shows a level of marker combination-expressing extracellular vesicles that is at least 30% or higher, including, e.g., at least 40%, at least 50%, at least 60%, at least 70%, at least 80%, at least 90%, at least 95% or higher, as compared to a reference threshold level. In some embodiments, a subject in need thereof is classified as having or being susceptible to cancer when the subject's biological sample material (e.g., a bodily fluid sample material, e.g., but not limited to, from a blood-derived sample) shows a level of marker combination-expressing extracellular vesicles that is at least 2-fold or higher, including, e.g., at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold, at least 250-fold, at least 500-fold, at least 750-fold, at least 1000-fold, or higher, as compared to a reference threshold level.

When a biological sample material (e.g., a bodily fluid sample material from, e.g., but not limited to, a blood-derived sample) from a subject in need thereof shows a comparable level to a reference threshold level, then the subject is classified as not likely to have or as not likely to be susceptible to cancer. In some such embodiments, a reference threshold level corresponds to a level of extracellular vesicles that express a marker combination in comparable samples from a population of reference subjects, e.g., non-cancer subjects. In some embodiments, exemplary non-cancer subjects include healthy subjects (e.g., healthy subjects of specified age ranges, such as e.g., below age 55 or above age 55), subjects with non-tumor related health diseases, disorders, or conditions (including, e.g., subjects having symptoms of cancerous diseases or disorders but not cancer), subjects having benign tumors, and combinations thereof.

In some embodiments, an oligo-linked probe as provided and/or utilized herein comprises a target-binding moiety and an oligonucleotide domain coupled to the target-binding moiety. In some embodiments, an oligonucleotide domain coupled to a target-binding moiety may comprise a double-stranded portion and a single-stranded overhang extended from at least one end of the oligonucleotide domain. In some embodiments, an oligonucleotide domain coupled to a target-binding moiety may comprise a double-stranded portion and a single-stranded overhang extended from each end of the oligonucleotide domain. In some embodiments, oligo-linked probes may be suitable for proximity-ligation-immuno quantitative polymerase chain reaction (pliq-PCR) and be referred to as pliq-PCR detection probes.

A target-binding moiety that is coupled to an oligonucleotide domain is an entity or an agent that specifically binds to a target (e.g., a provided marker of a marker combination; those skilled in the art will appreciate that, where the target marker is a particular form or moiety/component, the target-binding moiety specifically binds to that form or moiety/component). In some embodiments, a target-binding moiety may have a binding affinity (e.g., as measured by a dissociation constant) for a target (e.g., molecular target) of at least about 10⁻⁴M, at least about 10⁻⁵M, at least about 10⁻⁶M, at least about 10⁻⁷M, at least about 10⁻⁸M, at least about 10⁻⁹M, or lower. Those skilled in the art will appreciate that, in some cases, binding affinity (e.g., as measured by a dissociation constant) may be influenced by non-covalent intermolecular interactions such as hydrogen bonding, electrostatic interactions, hydrophobic and Van der Waals forces between the two molecules. Alternatively or additionally, binding affinity between a ligand and its target molecule may be affected by the presence of other molecules. Those skilled in the art will be familiar with a variety of technologies for measuring binding affinity and/or dissociation constants in accordance with the present disclosure, including, e.g., but not limited to ELISAs, surface plasmon resonance (SPR) assays, Luminex Single Antigen (LSA) assays, bio-layer interferometry (BLI) (e.g., Octet) assays, grating-coupled interferometry, and spectroscopic assays.

In some embodiments, a target-binding moiety is assessed for off-target effect. In some embodiments, a target-binding moiety is assessed using immunocapture followed by mass spectrometry (e.g., to reveal off target binding events in a complex sample). In some embodiments, a target-binding moiety is assessed using protein or glycan arrays, e.g., where many thousands of human proteins or glycans are arrayed on a chip and an antibody's binding is profiled across all available targets (e.g., a specific antibody will only bind to a target of interest). In some embodiments, a target-binding moiety is assessed using traditional immunoassays such as western blot. In some embodiments, a target-binding moiety is assessed for generic off-target non-specific binding (e.g., binding to other antibodies, DNA, lipids, etc.). In some embodiments, such generic off-target non-specific binding may be measured and identified using a negative control to identify a false positive signal (e.g., suggesting that one or more antibodies bind non-specifically, and not to a target).

In some embodiments, a target-binding moiety may be or comprise an agent of any chemical class such as, for example, a carbohydrate, a nucleic acid, a lipid, a metal, a polypeptide, a small molecule, etc., and/or a combination thereof. In some embodiments, a target-binding moiety may be or comprise an affinity agent such as an antibody, affimer, aptamer, lectin, siglec, etc. In some embodiments, a target-binding moiety is or comprises an antibody agent, e.g., an antibody agent that specifically binds to a target or an epitope thereof, e.g., a provided marker of a marker combination for cancer or an epitope thereof. In some embodiments, a target-binding moiety is or comprises a lectin or siglec that specifically binds to a carbohydrate-dependent marker as provided herein. In some embodiments, a target-binding moiety for a provided marker may be a commercially available. In some embodiments, a target-binding moiety for a provided marker may be designed and created for the purpose of use in assays as described herein. In some embodiments, a target-binding moiety is or comprises an aptamer, e.g., an aptamer that specifically binds to a target or an epitope thereof, e.g., a provided marker of a marker combination for cancer or an epitope thereof. In some embodiments, a target-binding moiety is or comprises an affimer molecule that specifically binds to a target or an epitope thereof, e.g., a provided marker of a marker combination for cancer or an epitope thereof. In some embodiments, such an affimer molecule can be or comprise a peptide or polypeptide that binds to a target or an epitope thereof (e.g., as described herein) with similar specificity and affinity to that of a corresponding antibody. In some embodiments, a target may be or comprise a target that is associated with cancer. For example, in some such embodiments, a cancer-associated target can be or comprise a target that is associated with more than one cancer (i.e., at least two or more cancers). In some embodiments, a cancer-associated target can be or comprise a target that is typically associated with cancers. In some embodiments, a cancer-associated target can be or comprise a target that is associated with cancers of a specific tissue, e.g., cancer. In some embodiments, a cancer-associated target can be or comprise a target that is specific to a particular cancer, e.g., a particular cancer and more specifically in some embodiments characterized by carcinoma, sarcoma, melanoma, and mixed types.

In some embodiments, a target-binding moiety recognizes and specifically binds to a target present in a biological entity (including, e.g., but not limited to cells and/or extracellular vesicles). For example, in some embodiments, a target-binding moiety may recognize and specifically bind to a tumor-associated antigen or epitope thereof. In some embodiments, a tumor-associated antigen may be or comprise an antigen that is associated with a cancer such as, for example, skin cancer, brain cancer (including, e.g., glioblastoma), breast cancer, liver cancer, lung cancer, ovarian cancer, pancreatic cancer, prostate cancer, skin cancer, etc. In some embodiments, a target-binding moiety may recognize a tumor antigen associated with cancer (e.g., in some embodiments characterized by carcinoma, sarcoma, melanoma, and mixed types). In some embodiments, a target-binding moiety may recognize a tumor antigen associated with in some embodiments characterized by carcinoma, sarcoma, melanoma, and mixed types.

In some embodiments, a target-binding moiety may specifically bind to an intravesicular target, e.g., a provided intravesicular protein or RNA (e.g., mRNA). In some embodiments, a target-binding moiety may specifically bind to a surface target that is present on/within extracellular vesicles, e.g., a membrane-bound polypeptide present on cancer-associated extracellular vesicles.

In some embodiments, a target-binding moiety is directed to a marker for a specific condition or disease (e.g., cancer), which marker is or has been determined, for example, by analyzing a population or library (e.g., tens, hundreds, thousands, tens of thousands, hundreds of thousands, or more) of patient biopsies and/or patient data to identify such a marker (e.g., a predictive marker).

In some embodiments, a relevant marker may be one identified and/or characterized, for example, via data analysis. In some embodiments, for example, a diverse set of data (e.g., in some embodiments comprising one or more of bulk RNA sequencing, single-cell RNA (scRNA) sequencing, mass spectrometry, histology, post-translational modification data, in vitro and/or in vivo experimental data) can be analyzed through machine learning and/or computational modeling to identify markers (e.g., predictive markers) that are highly specific to a disease or condition (e.g., cancer).

In some embodiments, a target-binding moiety is directed to a tissue-specific target, for example, a target that is associated with a specific tissue such as, for example, brain, breast, colon, ovary and/or other tissues associated with a female reproductive system, pancreas, prostate and/or other tissues associated with a male reproductive system, liver, lung, and skin. In some embodiments, such a tissue-specific target may be associated with a normal healthy tissue and/or a diseased tissue, such as a tumor. In some embodiments, a target-binding moiety is directed to a target that is specifically associated with a normal healthy condition of a subject. In some embodiments, a target-binding moiety may recognize a tissue specific antigen.

In some embodiments, individual target binding entities utilized in a plurality of oligo-linked probes (e.g., as described and/or utilized herein) are directed to different targets. In some embodiments, such different targets may represent different marker proteins or polypeptides. In some embodiments, such different targets may represent different epitopes of the same marker proteins or polypeptides. In some embodiments, two or more individual target binding entities utilized in a plurality of oligo-linked probes (e.g., as described and/or utilized herein) may be directed to the same target.

In some embodiments, individual target binding entities utilized in a plurality of oligo-linked probes for detection of cancer may be directed to different target markers of a marker combination for cancer.

In some embodiments, individual target binding entities utilized in a plurality of oligo-linked probes for detection of cancer may be directed to the same target marker of a marker combination for cancer. In some embodiments, such target binding entities may be directed to the same or different epitopes of the same target marker of such a marker combination for cancer.

In some embodiments, an oligonucleotide domain for use in accordance with the present disclosure (e.g., that may be coupled to a target-binding moiety) may comprise a double-stranded portion and a single-stranded overhang extended from one or both ends of the oligonucleotide domain. In some embodiments where an oligonucleotide domain comprises a single-stranded overhang extended from each end, a single-stranded overhang is extended from a different strand of a double-stranded portion. In some embodiments where an oligonucleotide domain comprises a single-stranded overhang extended from one end of the oligonucleotide domain, the other end of the oligonucleotide domain may be a blunt end.

In some embodiments, an oligonucleotide domain may comprise ribonucleotides, deoxyribonucleotides, synthetic nucleotide residues that are capable of participating in Watson-Crick type or analogous base pair interactions, and any combinations thereof. In some embodiments, an oligonucleotide domain is or comprises DNA. In some embodiments, an oligonucleotide domain is or comprises peptide nucleic acid (PNA).

In some embodiments, an oligonucleotide may have a length that is determined, at least in part, for example, by, e.g., the physical characteristics of an entity of interest (e.g., biological entity such as extracellular vesicles) to be detected, and/or selection and localization of molecular targets in an entity of interest (e.g., biological entity such as extracellular vesicles) to be detected. In some embodiments, an oligonucleotide domain of a oligo-linked probe is configured to have a length such that when a first oligo-linked probe and a second oligo-linked probe bind to an entity of interest (e.g., biological entity such as extracellular vesicles), the first single-stranded overhang and the second single-stranded overhang are in sufficiently close proximity to permit interaction (e.g., hybridization) between the single-stranded overhangs. For example, when an entity of interest (e.g., biological entity) is an extracellular vesicle (e.g., an exosome), oligonucleotide domains of oligo-linked probes can each independently have a length such that their respective single-stranded overhangs are in sufficiently close proximity to anneal or interact with each other when the corresponding oligo-linked probes are bound to the same extracellular vesicle. For example, in some embodiments, oligonucleotide domains of oligo-linked probes for use in detecting extracellular vesicles (e.g., an exosome) may each independently have a length of about 20 nm to about 200 nm, about 40 nm to about 500 nm, about 40 nm to about 300 nm, or about 50 nm to about 150 nm. In some embodiments, oligonucleotide domains of oligo-linked probes for use in detecting extracellular vesicles (e.g., an exosome) may each independently have a length of about 20 nm to about 200 nm. In some embodiments, lengths of oligonucleotide domains of oligo-linked probes in a set can each independently vary to increase and/or maximize the probability of them finding each other when they simultaneously bind to the same entity of interest. Such oligonucleotide domains designed for use in oligo-linked probes for detecting extracellular vesicles can also be used in oligo-linked probes for detecting extracellular vesicles.

Accordingly, in some embodiments, an oligonucleotide domain for use in technologies provided herein may have a length in the range of about 20 up to about 1000 nucleotides. In some embodiments, an oligonucleotide domain may have a length in the range of about 30 up to about 1000 nucleotides, In some embodiments, an oligonucleotide domain may have a length in the range of about 30 to about 500 nucleotides, from about 30 to about 250 nucleotides, from about 30 to about 200 nucleotides, from about 30 to about 150 nucleotides, from about 40 to about 150 nucleotides, from about 40 to about 125 nucleotides, from about 40 to about 100 nucleotides, from about 40 to about 60 nucleotides, from about 50 to about 90 nucleotides, from about 50 to about 80 nucleotides. In some embodiments, an oligonucleotide domain may have a length of at least 20 or more nucleotides, including, e.g., at least 30, at least 40, at least 50, at least 60, at least 70, at least 80, at least 90, at least 100, at least 250, at least 500, at least 750, at least 1000 nucleotides or more. In some embodiments, an oligonucleotide domain may have a length of no more than 1000 nucleotides or lower, including, e.g., no more than 900, no more than 800, no more than 700, no more than 600, no more than 500, no more than 400, no more than 300, no more than 200, no more than 100, no more than 90, no more than 80, no more than 70, no more than 60, no more than 50, no more than 40 nucleotides, no more than 30 nucleotides, no more than 20 nucleotides or lower.

In some embodiments, an oligonucleotide domain may have a length of about 20 nm to about 500 nm. In some embodiments, an oligonucleotide domain may have a length of about 20 nm to about 400 nm, about 30 nm to about 200 nm, about 50 nm to about 100 nm, about 30 nm to about 70 nm, or about 40 nm to about 60 nm. In some embodiments, an oligonucleotide domain may have a length of at least about 20 nm or more, including, e.g., at least about 30 nm, at least about 40 nm, at least about 50 nm, at least about 60 nm, at least about 70 nm, at least about 80 nm, at least about 90 nm, at least about 100 nm, at least about 200 nm, at least about 300 nm, at least about 400 nm or more. In some embodiments, an oligonucleotide domain may have a length of no more than 1000 nm or lower, including, e.g., no more than 900 nm, no more than 800 nm, no more than 700 nm, no more than 600 nm, no more than 500 nm, no more than 400 nm, no more than 300 nm, no more than 200 nm, no more than 100 nm or lower.

In some embodiments, a double-stranded portion of an oligonucleotide domain for use in technologies provided herein may have a length in the range of about 30 up to about 1000 nucleotides. In some embodiments, a double-stranded portion of an oligonucleotide domain may have a length in the range of about 30 to about 500 nucleotides, from about 30 to about 250 nucleotides, from about 30 to about 200 nucleotides, from about 30 to about 150 nucleotides, from about 40 to about 150 nucleotides, from about 40 to about 125 nucleotides, from about 40 to about 100 nucleotides, from about 50 to about 90 nucleotides, from about 50 to about 80 nucleotides. In some embodiments, a double-stranded portion of an oligonucleotide domain may have a length of at least 30 or more nucleotides, including, e.g., at least 40, at least 50, at least 60, at least 70, at least 80, at least 90, at least 100, at least 250, at least 500, at least 750, at least 1000 nucleotides or more. In some embodiments, a double-stranded portion of an oligonucleotide domain may have a length of no more than 1000 nucleotides or lower, including, e.g., no more than 900, no more than 800, no more than 700, no more than 600, no more than 500, no more than 400, no more than 300, no more than 200, no more than 100, no more than 90, no more than 80, no more than 70, no more than 60, no more than 50, no more than 40 nucleotides or lower.

In some embodiments, a double-stranded portion of an oligonucleotide domain may have a length of about 20 nm to about 500 nm. In some embodiments, a double-stranded portion of an oligonucleotide domain may have a length of about 20 nm to about 400 nm, about 30 nm to about 200 nm, about 50 nm to about 100 nm, about 30 nm to about 70 nm, or about 40 nm to about 60 nm. In some embodiments, a double-stranded portion of an oligonucleotide domain may have a length of at least about 20 nm or more, including, e.g., at least about 30 nm, at least about 40 nm, at least about 50 nm, at least about 60 nm, at least about 70 nm, at least about 80 nm, at least about 90 nm, at least about 100 nm, at least about 200 nm, at least about 300 nm, at least about 400 nm or more. In some embodiments, a double-stranded portion of an oligonucleotide domain may have a length of no more than 1000 nm or lower, including, e.g., no more than 900 nm, no more than 800 nm, no more than 700 nm, no more than 600 nm, no more than 500 nm, no more than 400 nm, no more than 300 nm, no more than 200 nm, no more than 100 nm or lower.

In some embodiments, a double-stranded portion of an oligonucleotide domain is characterized in that when oligo-linked probes are connected to each other through hybridization of respective complementary single-stranded overhangs (e.g., as described and/or utilized herein), the combined length of the respective oligonucleotide domains (including, if any, a linker that links a target-binding moiety to an oligonucleotide domain) is long enough to allow respective target binding entities to substantially span the full characteristic length (e.g., diameter) of an entity of interest (e.g., an extracellular vesicle). For example, in some embodiments where extracellular vesicles are entities of interest, a combined length of oligonucleotide domains (including, if any, a linker that links a target-binding moiety to an oligonucleotide domain) of oligo-linked probes may be approximately 50 to 200 nm, when the oligo-linked probes are fully connected to each other.

In some embodiments, a double-stranded portion of an oligonucleotide domain may comprise a binding site for a primer. In some embodiments, such a binding site for a primer may comprise a nucleotide sequence that is designed to reduce or minimize the likelihood for miss-priming or primer dimers. Such a feature, in some embodiments, can decrease the lower limit of detection and thus increase the sensitivity of systems provided herein. In some embodiments, a binding site for a primer may comprise a nucleotide sequence that is designed to have a similar annealing temperature as another primer binding site.

In some embodiments, a double-stranded portion of an oligonucleotide domain may comprise a nucleotide sequence designed to reduce or minimize overlap with nucleic acid sequences (e.g., DNA and/or RNA sequences) typically associated with genome and/or gene transcripts (e.g., genomic DNA and/or RNA, such as mRNA of genes) of a subject (e.g., a human subject). Such a feature, in some embodiments, may reduce or minimize interference of any genomic DNA and/or mRNA transcripts of a subject that may be present (e.g., as contaminants) in a sample during detection.

In some embodiments, a double-stranded portion of an oligonucleotide domain may have a nucleotide sequence designed to reduce or minimize formation of self-dimers, homo-dimers, or hetero-dimers.

In some embodiments, a single-stranded overhang of an oligonucleotide domain for use in technologies provided herein may have a length of about 2 to about 20 nucleotides. In some embodiments, a single-stranded overhang of an oligonucleotide domain may have a length of about 2 to about 15 nucleotides, from about 2 to about 10 nucleotides, from about 3 to about 20 nucleotides, from about 3 to about 15 nucleotides, from about 3 to about 10 nucleotides. In some embodiments, a single-stranded overhang can have at least 1 to 5 nucleotides in length. In some embodiments, a single-stranded overhang of an oligonucleotide domain may have a length of at least 2 or more nucleotides, including, e.g., at least 3, at least 4, at least 5, at least 6, at least 7, at least 8, at least 9, at least 10, at least 11, at least 12, at least 13, at least 14, at least 15, at least 20 nucleotides, or more. In some embodiments, a single-stranded overhang of an oligonucleotide domain may have a length of no more than 20 nucleotides or lower, including, e.g., no more than 15, no more than 14, no more than 13, no more than 12, no more than 11, no more than 10, no more than 9, no more than 8, no more than 7, no more than 6, no more than 5, no more than 4 nucleotides or lower.

In some embodiments, a single-stranded overhang of an oligonucleotide domain may have a length of about 1 nm to about 10 nm. In some embodiments, a single-stranded overhang of an oligonucleotide domain may have a length of about 1 nm to about 5 nm. In some embodiments, a single-stranded overhang of an oligonucleotide domain may have a length of at least about 0.5 nm or more, including, e.g., at least about 1 nm, at least about 1.5 nm, at least about 2 nm, at least about 3 nm, at least about 4 nm, at least about 5 nm, at least about 6 nm, at least about 7 nm, at least about 8 nm, at least about 9 nm, at least about 10 nm or more. In some embodiments, a single-stranded overhang of an oligonucleotide domain may have a length of no more than 10 nm or lower, including, e.g., no more than 9 nm, no more than 8 nm, no more than 7 nm, no more than 6 nm, no more than 5 nm, no more than 4 nm, no more than 3 nm, no more than 2 nm, no more than 1 nm or lower.

A single-stranded overhang of an oligonucleotide domain is designed to comprise a nucleotide sequence that is complementary to at least a portion of a single-stranded overhang of a second oligo-linked probe such that a double-stranded complex comprising a first oligo-linked probe and a second oligo-linked probe can be formed through hybridization of the complementary single-stranded overhangs. In some embodiments, nucleotide sequences of complementary single-stranded overhangs are selected for optimal ligation efficiency in the presence of an appropriate nucleic acid ligase. In some embodiments, a single-stranded overhang has a nucleotide sequence preferentially selected for efficient ligation by a specific nucleic acid ligase of interest (e.g., a DNA ligase such as a T4 or T7 ligase). For example, such a single-stranded overhang may have a nucleotide sequence of GAGT, e.g., as described in Song et al., “Enzyme-guided DNA sewing architecture” Scientific Reports 5: 17722 (2015), which is incorporated herein by reference for the purpose described herein. In other examples, blunt-end ligation is used.

When two oligo-linked probes couple together through hybridization of respective complementary single-stranded overhangs, their respective oligonucleotide domains comprising the hybridized single-stranded overhangs can, in some embodiments, have a combined length of about 90%-110% or about 95%-105% of a characteristic length (e.g., diameter) of an entity of interest (e.g., a biological entity). For example, in some embodiments when a biological entity is an exosome, the combined length can be about 50 nm to about 200 nm, or about 75 nm to about 150 nm, or about 80 nm to about 120 nm.

An oligonucleotide domain and a target-binding moiety can be coupled together in a oligo-linked probe by a covalent linkage, and/or by a non-covalent association (such as, e.g., a protein-protein interaction such as streptavidin-biotin interaction and/or an ionic interaction). In some embodiments, a oligo-linked probe appropriate for use in accordance with the present disclosure is a conjugate molecule comprising a target-binding moiety and an oligonucleotide domain, where the two components are typically covalently coupled to each other, e.g., directly through a bond, or indirectly through one or more linkers. In some embodiments, a target-binding moiety is coupled to one of two strands of an oligonucleotide domain by a covalent linkage (e.g., directly through a bond or indirectly through one or more linkers) and/or by a non-covalent association (such as, e.g., a protein-protein interaction such as streptavidin-biotin interaction and/or ionic interaction).

Where linkers are employed, in some embodiments, linkers are chosen to provide for covalent attachment of a target-binding moiety to one or both strands of an oligonucleotide domain through selected linkers. In some embodiments, linkers are chosen such that the resulting covalent attachment of a target-binding moiety to one or both strands of an oligonucleotide domain maintains the desired binding affinity of the target-binding moiety for its target. In some embodiments, linkers are chosen to enhance binding specificity of a target-binding moiety for its target. Linkers and/or conjugation methods of interest may vary widely depending on a target-binding moiety, e.g., its size and/or charges. In some embodiments, linkers are biologically inert.

A variety of linkers and/or methods for coupling a target-binding moiety to an oligonucleotide is known to one of ordinary skill in the art and can be used in accordance with the present disclosure. In some embodiments, a linker can comprise a spacer group at either end with a reactive functional group at either end capable of covalent attachment to a target-binding moiety. Examples of spacer groups that can be used in linkers include, but are not limited to, aliphatic and unsaturated hydrocarbon chains (including, e.g., C4, C5, C6, C7, C8, C9, C10, C11, C12, C13, C14, C15, C16, C17, C18, C19, C20, or longer), spacers containing heteroatoms such as oxygen (e.g., ethers such as polyethylene glycol) or nitrogen (polyamines), peptides, carbohydrates, cyclic or acyclic systems that may contain heteroatoms. Non-limiting examples of a reactive functional group to facilitate covalent attachment include nucleophilic functional groups (e.g., amines, alcohols, thiols, and/or hydrazides), electrophilic functional groups (e.g., aldehydes, esters, vinyl ketones, epoxides, isocyanates, and/or maleimides), functional groups capable of cycloaddition reactions, forming disulfide bonds, or binding to metals. In some embodiments, exemplary reactive functional groups, but are not limited to, primary and secondary amines, hydroxamic acids, N-hydroxysuccinimidyl (NHS) esters, dibenzocyclooctyne (DBCO)-NHS esters, azido-NHS esters, azidoacetic acid NHS ester, propargyl-NHS ester, trans-cyclooctene-NHS esters, N-hydroxysuccinimidyl carbonates, oxycarbonylimidazoles, nitrophenylesters, trifluoroethyl esters, glycidyl ethers, vinylsulfones, maleimides, azidobenzoyl hydrazide, N-[4-(p-azidosalicylamino)butyl]-3′-[2′-pyridyldithio]propionamid), bis-sulfosuccinimidyl suberate, dimethyladipimidate, disuccinimidyltartrate, N-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, and succinimidyl 4-[N-maleimidomethyl]cyclohexane-1-carboxylate, 3-(2-pyridyldithio)propionic acid N-hydroxysuccinimide ester (SPDP), 4-(N-maleimidomethyl)-cyclohexane-1-carboxylic acid N-hydroxysuccinimide ester (SMCC), and any combinations thereof.

In some embodiments, a target-binding moiety (e.g., a target binding antibody agent) is coupled or conjugated to one or both strands of an oligonucleotide domain using N-hydrosysuccinimide (NHS) ester chemistry. NHS esters react with free primary amines and result in stable covalent attachment. In some embodiments, a primary amino group can be positioned at a terminal end with a spacer group, e.g., but not limited to an aliphatic and unsaturated hydrocarbon chain (e.g., a C6 or C12 spacer group).

In some embodiments, a target-binding moiety (e.g., a target-binding affinity agent) can be coupled or conjugated to one or both strands of an oligonucleotide domain using a site-specific conjugation method known in the art, e.g., to enhance the binding specificity of conjugated target-binding moiety (e.g., conjugated target-binding affinity agent). Examples of a site-specific conjugation method include, but are not limited to coupling or conjugation through a disulfide bond, C-terminus, carbohydrate residue or glycan, and/or unnatural amino acid labeling. In some embodiments where a target-binding moiety is or comprises an affinity agent, an oligonucleotide can be coupled or conjugated to the target-binding moiety via at least one or more free amine groups present in the target-binding moiety. In some embodiments, an oligonucleotide can be coupled or conjugated to a target-binding moiety that is or comprises an affinity agent via at least one or more reactive thiol groups present in the target-binding moiety. In some embodiments, an oligonucleotide can be coupled or conjugated to a target-binding moiety that is or comprises an antibody agent or a peptide aptamer via at least one or more carbohydrate residues present in the target-binding moiety.

In some embodiments, a plurality of oligonucleotides (e.g., at least 2, at least 3, at least 4, at least 5, at least 6, at least 7, at least 8, at least 9, at least ten, or more) can be coupled or conjugated to a target-binding moiety (e.g., a target binding antibody agent).

In some embodiments, at least one or more marker combinations that are suitable for detection of Adrenocortical carcinoma (ACC) can be included in cancer detection. In some embodiments, marker combinations can enrich a population for subjects who may likely be suffering from or be susceptible to ACC.

In some embodiments, a marker combination suitable for enriching a population for subjects who may be likely suffering from or be likely susceptible to ACC comprises at least three markers, selected from the group consisting of: a CDH2 polypeptide, a ENPP5 polypeptide, and a RNF128 polypeptide; or a CDH2 polypeptide, a LAPTM4B polypeptide, and a PODXL2 polypeptide; or a APOO polypeptide, a GJB1 polypeptide, and a IGSF3 polypeptide; or a CDH2 polypeptide, a FERMT1 polypeptide, and a NPTXR polypeptide; or a CDH2 polypeptide, a CLDN3 polypeptide, and a PRAF2 polypeptide; or combinations thereof. In some embodiments, any two markers of the 3-biomarker combinations as described herein for detection of ACC can be used as a 2-marker combination for detection of ACC.

In some embodiments, at least one or more marker combinations that are suitable for detection of Bladder Urothelial Carcinoma (BLCA) can be included in cancer detection. In some embodiments, marker combinations can enrich a population for subjects who may likely be suffering from or be susceptible to BLCA.

In some embodiments, a marker combination suitable for enriching a population for subjects who may be likely suffering from or be likely susceptible to BLCA comprises at least three markers, selected from the group consisting of: a AP1M2 polypeptide, a HS6ST2 polypeptide, and a ULBP2 polypeptide; or a B3GNT3 polypeptide, a LAMB3 polypeptide, and a ULBP2 polypeptide; or a B3GNT3 polypeptide, a CDH3 polypeptide, and a FZD2 polypeptide; or a CDH1 polypeptide, a HS6ST2 polypeptide, and a ULBP2 polypeptide; or a HS6ST2 polypeptide, a LSR polypeptide, and a PODXL2 polypeptide; or a FERMT1 polypeptide, a HS6ST2 polypeptide, and a ULBP2 polypeptide; or a ILDR1 polypeptide, a PODXL2 polypeptide, and a ULBP2 polypeptide; or a B3GNT3 polypeptide, a LAMC2 polypeptide, and a ULBP2 polypeptide; or a AP1M2 polypeptide, a HS6ST2 polypeptide, and a PODXL2 polypeptide; or a AP1M2 polypeptide, a HS6ST2 polypeptide, and a KPNA2 polypeptide; or a CDH3 polypeptide, a EPHB2 polypeptide, and a LAMC2 polypeptide; or a HS6ST2 polypeptide, a LAMB3 polypeptide, and a ULBP2 polypeptide; or a FZD2 polypeptide, a PODXL2 polypeptide, and a SMIM22 polypeptide; or a LAMC2 polypeptide, a LSR polypeptide, and a ULBP2 polypeptide; or a B3GNT3 polypeptide, a FZD2 polypeptide, and a LAMC2 polypeptide; or combinations thereof. In some embodiments, any two markers of the 3-biomarker combinations as described herein for detection of BLCA can be used as a 2-marker combination for detection of BLCA.

In some embodiments, at least one or more marker combinations that are suitable for detection of Brain Lower Grade Glioma (LGG) can be included in cancer detection. In some embodiments, marker combinations can enrich a population for subjects who may likely be suffering from or be susceptible to LGG.

In some embodiments, a marker combination suitable for enriching a population for subjects who may be likely suffering from or be likely susceptible to LGG comprises at least three markers, selected from the group consisting of: a CDH2 polypeptide, a FERMT1 polypeptide, and a LRRN1 polypeptide; or a CDH2 polypeptide, a LRRN1 polypeptide, and a SLC4A4 polypeptide; or a CDH2 polypeptide, a LRRN1 polypeptide, and a PRAF2 polypeptide; or a CDH2 polypeptide, a LRRN1 polypeptide, and a PODXL2 polypeptide; or a CDH2 polypeptide, a GPRIN1 polypeptide, and a LRRN1 polypeptide; or a CDH2 polypeptide, a PODXL2 polypeptide, and a SLC4A4 polypeptide; or a CDH2 polypeptide, a LRRN1 polypeptide, and a NPTXR polypeptide; or a CDH2 polypeptide, a LRRN1 polypeptide, and a TMEM132A polypeptide; or a CDH2 polypeptide, a GOLM1 polypeptide, and a LRRN1 polypeptide; or a CDH2 polypeptide, a FERMT1 polypeptide, and a NPTXR polypeptide; or a CDH2 polypeptide, a LRRN1 polypeptide, and a RAC3 polypeptide; or a CADM4 polypeptide, a CDH2 polypeptide, and a FERMT1 polypeptide; or a CADM4 polypeptide, a GPRIN1 polypeptide, and a LRRN1 polypeptide; or a CADM4 polypeptide, a CDH2 polypeptide, and a LRRN1 polypeptide; or a CDH2 polypeptide, a GNG4 polypeptide, and a LRRN1 polypeptide; or combinations thereof. In some embodiments, any two markers of the 3-biomarker combinations as described herein for detection of LGG can be used as a 2-marker combination for detection of LGG.

In some embodiments, at least one or more marker combinations that are suitable for detection of breast cancer can be included in cancer detection. In some embodiments, provided marker combinations can enrich a population for subjects who may likely be suffering from or be susceptible to breast cancer.

In some embodiments, one or more markers that are suitable for detection of breast cancer and are useful to be included in cancer detection can be selected from: (i) polypeptides encoded by human genes as follows: ABCC11, ABCD3, ACSL3, ALCAM, ALDH18A1, AP1M2, AP2B1, APOO, APP, ARFGEF3, ATP6AP2, BROX, BSPRY, CA12, CALU, CANT1, CANX, CDH1, CDH3, CELSR1, CELSR2, CIP2A, CLGN, CLN5, CLSTN2, CLTC, CNNM4, COPA, COX6C, DAG1, DNAJC1, DSC2, DSG2, DSG3, EFHD1, EGFR, ENPP1, EPCAM, EPHB3, EPPK1, ERBB2, ERBB3, ERBB4, ERMP1, ESR1, FAM120A, FGFR4, FUT8, GALNT3, GALNT6, GALNT7, GBP5, GDAP1, GDI2, GFRA1, GNPNAT1, GOLM1, GOLPH3L, GPRIN1, GRB7, GRHL2, HACD3, HID1, IGF1R, ITGA11, ITGB6, ITPR2, KCTD3, KIF16B, KIF1A, KPNA2, LAMC2, LAMP2, LAMTOR2, LANCL2, LMNB1, LRBA, LRP2, LRRC59, LSR, MAGI3, MAP7, MAPT, MARCKSL1, MEAK7, MELK, MIEN1, MTCH2, MUC1, MYO6, NCAM2, NECTIN2, NECTIN4, NUCB2, NUP155, NUP210, OCLN, PARD6B, PDIA6, PIGT, PLEKHF2, PLGRKT, PLOD1, PREX1, PROM1, PTK7, PTPRF, PTPRK, QSOX1, RAB25, RAB27B, RAB30, RABEP1, RAC3, RACGAP1, RAP2B, RCC2, REEP6, RPN1, SCUBE2, SEC23B, SEPHS1, SFXN2, SHROOM3, SIPAIL3, SLC1A4, SLC35B2, SLC9A3R1, SPTLC2, SSR1, ST14, STARD10, STX6, SUMO1, SYAP1, SYT7, SYTL2, TACSTD2, TJP3, TMED2, TMED3, TMEM132A, TMEM87B, TMPO, TOM1L1, TOMM34, TRAF4, YES1, ZMPSTE24, ADAM8, CCL8, CCN1, CCR5, CD274, CD44, CDH11, CSPG4, DLL4, EPHA10, FGF1, FLNA, FZD7, GPNMB, IL1RAP, ITGA6, LY6E, MCAM, MELTF, MERTK, MUC16, NRP1, NT5E, PRLR, RET, S1PR1, SLC39A6, SLC3A2, SLC7A11, SLC7A5, STAT3, SUSD3, TF, TMPRSS1, TNC, TNFRSF12A, VANGL2, VEGFA, VTCN1, XBP1, and combinations thereof; and/or (ii) carbohydrate-dependent markers as follows: CA15-3 antigen, CA27-29 antigen, Phosphatidylserine, Tn antigen, SialylTn (sTn) antigen, Thomsen-Friedenreich (T, TF) antigen, Lewis Y antigen (also known as CD174), Sialyl Lewis X (sLex) antigen (also known as Sialyl SSEA-1 (SLX)), Sialyl Lewis A antigen (also known as CA19-9), SSEA-1 (also known as Lewis X antigen), NeuGcGM3 (N-glycolyl GM3 ganglioside), and combinations thereof.

In some embodiments, one or more markers that are suitable for detection of breast cancer and are useful to be included in cancer detection can be selected from: (i) polypeptides encoded by human genes as follows: ABCC11, AP1M2, APOO, ARFGEF3, BSPRY, CANT1, CDH1, CDH3, CELSR1, CIP2A, CLGN, COX6C, DSC2, DSG2, EGFR, EPCAM, EPHB3, ERBB2, ERBB3, ESR1, FGFR4, FUT8, GALNT3, GALNT6, GALNT7, GFRA1, GOLM1, GRB7, GRHL2, HACD3, ITGB6, KIF1A, KPNA2, LAMC2, LMNB1, LRP2, LSR, MARCKSL1, MIEN1, MUC1, NECTIN2, NUP155, NUP210, OCLN, PARD6B, PLEKHF2, PRLR, PROM1, PTK7, PTPRK, RAB25, RAB27B, RAC3, SEPHS1, SFXN2, SHROOM3, SLC35B2, SLC9A3R1, ST14, SYT7, TJP3, TMEM132A, XBP1, and combinations thereof; and/or (ii) carbohydrate-dependent markers as follows: Lewis Y antigen (also known as CD174), Sialyl Lewis X (sLex) antigen (also known as Sialyl SSEA-1 (SLX)), T antigen, Tn antigen, and combinations thereof.

In some embodiments, at least one or more marker combinations that are suitable for detection of Breast invasive carcinoma (BRCA) can be included in cancer detection. In some embodiments, marker combinations can enrich a population for subjects who may likely be suffering from or be susceptible to BRCA.

In some embodiments, a marker combination suitable for enriching a population for subjects who may be likely suffering from or be likely susceptible to BRCA comprises at least three markers, selected from the group consisting of: a GALNT6 polypeptide, a SLC39A6 polypeptide, and a SMIM22 polypeptide; or a PARD6B polypeptide, a SLC39A6 polypeptide, and a SMIM22 polypeptide; or a MARCKSL1 polypeptide, a SLC39A6 polypeptide, and a SMIM22 polypeptide; or a APOO polypeptide, a SLC39A6 polypeptide, and a SMIM22 polypeptide; or a SLC39A6 polypeptide, a SMIM22 polypeptide, and a TSPAN13 polypeptide; or a SLC39A6 polypeptide, a SMIM22 polypeptide, and a SYAP1 polypeptide; or a CANT1 polypeptide, a SLC39A6 polypeptide, and a SMIM22 polypeptide; or a FAM241B polypeptide, a SLC39A6 polypeptide, and a SMIM22 polypeptide; or a ELAPOR1 polypeptide, a MARCKSL1 polypeptide, and a SLC39A6 polypeptide; or a ARFGEF3 polypeptide, a SLC39A6 polypeptide, and a SMIM22 polypeptide; or a AP1M2 polypeptide, a GPR160 polypeptide, and a SLC39A6 polypeptide; or a SLC39A6 polypeptide, a SMIM22 polypeptide, and a TMEM9 polypeptide; or a ILDR1 polypeptide, a MARCKSL1 polypeptide, and a SLC39A6 polypeptide; or a PODXL2 polypeptide, a SLC39A6 polypeptide, and a SMIM22 polypeptide; or a SHISA2 polypeptide, a SLC39A6 polypeptide, and a SLC44A4 polypeptide; or combinations thereof. In some embodiments, any two markers of the 3-biomarker combinations as described herein for detection of breast cancer can be used as a 2-marker combination for detection of breast cancer.

In some embodiments, at least one or more marker combinations that are suitable for detection of Endocervical Adenocarcinoma (CESC) can be included in cancer detection. In some embodiments, marker combinations can enrich a population for subjects who may likely be suffering from or be susceptible to CESC.

In some embodiments, a marker combination suitable for enriching a population for subjects who may be likely suffering from or be likely susceptible to CESC comprises at least three markers, selected from the group consisting of: a ILDR1 polypeptide, a PODXL2 polypeptide, and a ULBP2 polypeptide; or a HS6ST2 polypeptide, a LAMB3 polypeptide, and a LMNB1 polypeptide; or a HS6ST2 polypeptide, a LAMB3 polypeptide, and a ULBP2 polypeptide; or a AP1M2 polypeptide, a HS6ST2 polypeptide, and a ULBP2 polypeptide; or a CDH1 polypeptide, a HS6ST2 polypeptide, and a ULBP2 polypeptide; or a HS6ST2 polypeptide, a LAMC2 polypeptide, and a ULBP2 polypeptide; or a HS6ST2 polypeptide, a LAMC2 polypeptide, and a LMNB1 polypeptide; or a ILDR1 polypeptide, a LAMC2 polypeptide, and a RAP2B polypeptide; or a HS6ST2 polypeptide, a LAMC2 polypeptide, and a RAP2B polypeptide; or a HS6ST2 polypeptide, a LAMC2 polypeptide, and a RACGAP1 polypeptide; or a HS6ST2 polypeptide, a LAMB3 polypeptide, and a RACGAP1 polypeptide; or a HS6ST2 polypeptide, a LAMB3 polypeptide, and a LAMC2 polypeptide; or a HS6ST2 polypeptide, a LMNB1 polypeptide, and a LSR polypeptide; or a LAMC2 polypeptide, a LSR polypeptide, and a ULBP2 polypeptide; or a CDH3 polypeptide, a EPCAM polypeptide, and a ULBP2 polypeptide; or combinations thereof. In some embodiments, any two markers of the 3-biomarker combinations as described herein for detection of CESC can be used as a 2-marker combination for detection of CESC.

In some embodiments, at least one or more marker combinations that are suitable for detection of Cholangiocarcinoma (CHOL) can be included in cancer detection. In some embodiments, marker combinations can enrich a population for subjects who may likely be suffering from or be susceptible to CHOL.

In some embodiments, one or more markers that are suitable for detection of CHOL and are useful to be included in cancer detection can be selected from: (i) polypeptides encoded by human genes as follows: ANXA13, AQP1, ASPHD1, ATP1B1, B3GNT3, CDC42EP1, CDH1, CDH2, CFTR, CHST4, CLDN1, CLDN10, CLDN9, CLTRN, CPNE7, CRB3, DEFB1, EFNA4, EPCAM, FAM71A1, FAM241B, FGFR2, FGFR4, FRAS1, GAL3ST1, GGT1, GJB1, GRID1, HKDC1, HPN, IGSF3, KRTCAP3, LAD1, LAMC2, LPAR2, LSR, LYPD1, LYPD6B, MAL2, MARVELD2, MMP15, MPC2, MPP6, MUC1, MUC2, MUC4, MUC5AC, NCEH1, NRSN2, OCLN, OXTR, PARD6B, PDGFC, PIGT, PIK3AP1, PMEPA1, RAB25, RHOV, SHANK2, SLC39A6, SLC44A3, SLC4A4, SLC52A3, SMIM22, SNAP25, SYT13, TESC, TGFA, TM4SF4, TMCO1, TMEM132A, TMEM156, TMPRSS13, TNFRSF12A, TNFRSF21, TOMM20, UGT2A3, VEPH1, VTCN1, and combinations thereof; and/or (ii) carbohydrate-dependent markers as follows: Lewis Y antigen (also known as CD174), Tn antigen, Thomsen-Friedenreich (T, TF) antigen, Sialyl Lewis X (sLex) antigen (also known as Sialyl SSEA-1 (SLX)), and combinations thereof.

In some embodiments, one or more markers that are suitable for detection of CHOL and are useful to be included in cancer detection can be selected from: (i) polypeptides encoded by human genes as follows: ANXA13, ASPHD1, B3GNT3, CDH1, CDH2, CHST4, CLDN10, CLTRN, DEFB1, FGFR4, GAL3ST1, GJB1, HKDC1, IGSF3, KRTCAP3, LAD1, LAMC2, LSR, LYPD6B, MUC1, MUC2, MUC4, MUC5AC, OXTR, PIK3AP1, SHANK2, SLC44A3, SNAP25, SYT13, TESC, TM4SF4, TMEM156, VEPH1, VTCN1, and combinations thereof; and/or (ii) carbohydrate-dependent markers as follows: Lewis Y antigen (also known as CD174), Sialyl Lewis X (sLex) antigen (also known as Sialyl SSEA-1 (SLX)), T antigen, Tn antigen, and combinations thereof.

In some embodiments, a marker combination suitable for enriching a population for subjects who may be likely suffering from or be likely susceptible to CHOL comprises at least three markers, selected from the group consisting of: a PARD6B polypeptide, a PMEPA1 polypeptide, and a SYT13 polypeptide; or a B3GNT3 polypeptide, a LAMC2 polypeptide, and a PMEPA1 polypeptide; or a CDH1 polypeptide, a CDH2 polypeptide, and a LAMC2 polypeptide; or a CDH2 polypeptide, a ILDR1 polypeptide, and a LAMC2 polypeptide; or a CYP2S1 polypeptide, a SYT13 polypeptide, and a VTCN1 polypeptide; or a CDH1 polypeptide, a CDH2 polypeptide, and a ILDR1 polypeptide; or a PARD6B polypeptide, a SLC39A6 polypeptide, and a SYT13 polypeptide; or a CDH2 polypeptide, a CLN5 polypeptide, and a SYT13 polypeptide; or a CDH2 polypeptide, a FAM241B polypeptide, and a ILDR1 polypeptide; or a CDH2 polypeptide, a ILDR1 polypeptide, and a MAL2 polypeptide; or a CDH2 polypeptide, a CLN5 polypeptide, and a EPCAM polypeptide; or a APOO polypeptide, a GJB1 polypeptide, and a IGSF3 polypeptide; or a CDH2 polypeptide, a ILDR1 polypeptide, and a RAP2B polypeptide; or a CDH2 polypeptide, a EPCAM polypeptide, and a RCC2 polypeptide; or a CDH2 polypeptide, a ILDR1 polypeptide, and a RCC2 polypeptide; or combinations thereof. In some embodiments, any two markers of the 3-biomarker combinations as described herein for detection of CHOL can be used as a 2-marker combination for detection of CHOL.

In some embodiments, at least one or more marker combinations that are suitable for detection of Colon Adenocarcinoma (COAD) can be included in cancer detection. In some embodiments, marker combinations can enrich a population for subjects who may likely be suffering from or be susceptible to COAD.

In some embodiments, one or more markers that are suitable for detection of COAD and are useful to be included in cancer detection can be selected from: (i) polypeptides encoded by human genes as follows: ACSL5, ACVR2B, ALDH18A1, ALG5, AP1M2, ATP1B1, B3GNT3, BCAP31, CASK, CD133, CDH1, CDH17, CDH3, CEACAM5, CEACAM6, CFB, CFTR, CHDH, CHMP4B, CISD2, CLDN3, CLDN4, CLIC1, COPG2, CYP2S1, DPEP1, DSG2, EDAR, EPCAM, EPHB2, EPHB3, ERMP1, FAM241B, FERMT1, GALNT3, GNPNAT1, GOLIM4, GPA33, GPCRSA, HACD3, HEPH, HKDC1, HS6ST2, IHH, ILDR1, ITGA2, KCNQ1, KEL, KPNA2, LAD1, LAMC2, LBR, LMNB1, LMNB2, LSR, MAP7, MARCKSL1, MARVELD2, MGAT5, MLEC, MUC1, MUC13, NCEH1, NDUFS6, NLN, NOX1, NUP210, OCIAD2, PGAMS, PIGR, PIGT, PLEK2, PMEPA1, PTK7, RAB25, RAP2A, RAP2B, RCC2, RNF43, RPN1, RPN2, RPS3, RUVBL2, S100P, SLC12A2, SLC25A6, SLC2A1, SMIM22, SNTBI, SORD, SSR4, ST14, STOML2, STT3B, SYAP1, TESC, TM9SF2, TMED2, TMPO, TOMM22, TOMM34, AMHR2, CLDN1, DLL4, EGFR, ERBB2, FAP, FGFR4, FOLR1, GUCY2C, IGF1R, IL1A, ITGAV, KRT8, LGR5, LPR6, MET, MST1R, MUC5AC, TNFRSF10B, VEGFA, and combinations thereof; and/or (ii) carbohydrate-dependent markers as follows: CanAg (glycoform of MUC1), Lewis Y/B antigen, Lewis B Antigen, Sialyltetraosyl carbohydrate, Tn antigen, SialylTn (sTn) antigen, Thomsen-Friedenreich (T, TF) antigen, Lewis Y antigen (also known as CD174), Sialyl Lewis X (sLex) antigen (also known as Sialyl SSEA-1 (SLX)), Sialyl Lewis A antigen (also known as CA19-9), SSEA-1 (also known as Lewis X antigen), NeuGcGM3, and combinations thereof.

In some embodiments, one or more markers that are suitable for detection of COAD and are useful to be included in cancer detection can be selected from: (i) polypeptides encoded by human genes as follows: ACVR2B, B3GNT3, CD133, CDH17, CDH3, CEACAM5, CEACAM6, CFB, CFTR, CYP2S1, DLL4, EDAR, EPCAM, EPHB2, EPHB3, ERBB2, FAP, GPCRSA, IHH, ILDR1, ITGAV, KCNQ1, KEL, MARCKSL1, MSTIR, MUC1, MUCSAC, NOX1, OCIAD2, RNF43, SMIM22, and combinations thereof; and/or (ii) carbohydrate-dependent markers as follows: Lewis Y antigen (also known as CD174), SialylTn (sTn) antigen, Sialyl Lewis X (sLex) antigen (also known as Sialyl SSEA-1 (SLX)), T antigen, Tn antigen, and combinations thereof.

In some embodiments, a marker combination suitable for enriching a population for subjects who may be likely suffering from or be likely susceptible to COAD comprises at least three markers, selected from the group consisting of: a CDH17 polypeptide, a CDH3 polypeptide, and a FERMT1 polypeptide; or a CDH17 polypeptide, a CDH3 polypeptide, and a GALNT6 polypeptide; or a CDH17 polypeptide, a CDH3 polypeptide, and a CYP2S1 polypeptide; or a CDH17 polypeptide, a CDH3 polypeptide, and a PMEPA1 polypeptide; or a CDH17 polypeptide, a CDH3 polypeptide, and a MARCKSL1 polypeptide; or a CDH17 polypeptide, a CDH3 polypeptide, and a RNF128 polypeptide; or a CDH17 polypeptide, a CDH3 polypeptide, and a PODXL2 polypeptide; or a CDH3 polypeptide, a CYP2S1 polypeptide, and a GJB1 polypeptide; or a CDH3 polypeptide, a CLDN3 polypeptide, and a CYP2S1 polypeptide; or a CDH3 polypeptide, a CYP2S1 polypeptide, and a EPCAM polypeptide; or a CDH3 polypeptide, a CEACAM5 polypeptide, and a FERMT1 polypeptide; or a CDH3 polypeptide, a CEACAM6 polypeptide, and a EPHB2 polypeptide; or a CDH3 polypeptide, a CEACAM5 polypeptide, and a CYP2S1 polypeptide; or a CDH3 polypeptide, a CEACAM5 polypeptide, and a CLN5 polypeptide; or a CDH3 polypeptide, a CEACAM5 polypeptide, and a PODXL2 polypeptide; or combinations thereof. In some embodiments, any two markers of the 3-biomarker combinations as described herein for detection of COAD can be used as a 2-marker combination for detection of COAD.

In some embodiments, at least one or more marker combinations that are suitable for detection of Esophageal Carcinoma (ESCA) can be included in cancer detection. In some embodiments, marker combinations can enrich a population for subjects who may likely be suffering from or be susceptible to ESCA.

In some embodiments, one or more markers that are suitable for detection of ESCA and are useful to be included in cancer detection can be selected from: (i) polypeptides encoded by human genes as follows: ABCA12, ABCC1, ANO1, AP1S3, B3GNT3, CD24, CDCP1, CDH1, CDH17, CDH3, CEACAM5, CEACAM6, CELSR1, CLCA2, CREB3L1, CYP2S1, CYP4F11, DSC2, DSG2, DSG3, EPCAM, EPHB2, EPPK1, FAT1, FAT2, FERMT1, FUT2, GALNT3, GALNT5, GCNT3, GJB2, HAS3, HS6ST2, ITGA2, ITGB6, JUP, KDELR3, KPNA2, LAD1, LAMB3, LAMC2, LAMP3, LAPTM4B, LSR, MAL2, MARVELD2, MET, MGAM2, MUC1, MUC13, MUC4, NCEH1, NECTIN1, PANX2, PHLDA2, PIGT, PMEPA1, PRR7, PRSS21, PTPRH, RNF128, SLC7A11, SLC7A5, TACSTD2, TENM2, TGFA, TMC5, TMEM132A, TMEM158, TMPRSS11D, TMPRSS4, TNFRSF21, TOR4A, TTYH3, UGT8, ULBP2, and combinations thereof; and/or (ii) carbohydrate-dependent markers as follows: Lewis Y antigen (also known as CD174), Tn antigen, SialylTn (sTn) antigen, Thomsen-Friedenreich (T, TF) antigen, Sialyl Lewis X (sLex) antigen (also known as Sialyl SSEA-1 (SLX)), and combinations thereof.

In some embodiments, one or more markers that are suitable for detection of ESCA and are useful to be included in cancer detection can be selected from: (i) polypeptides encoded by human genes as follows: ANO1, AP1S3, B3GNT3, CDCP1, CEACAM5, CEACAM6, CELSR1, CLCA2, CYP2S1, CYP4F11, DSC2, DSG2, DSG3, EPCAM, EPPK1, GALNT3, HS6ST2, ITGB6, LAMB3, LAMC2, LSR, MAL2, MARVELD2, MUC1, MUC13, PRR7, SLC7A5, TMEM158, TMPRSS11D, UGT8, ULBP2, and combinations thereof; and/or (ii) carbohydrate-dependent markers as follows: Lewis Y antigen (also known as CD174), SialylTn (sTn) antigen, Sialyl Lewis X (sLex) antigen (also known as Sialyl SSEA-1 (SLX)), T antigen, Tn antigen, and combinations thereof.

In some embodiments, a marker combination suitable for enriching a population for subjects who may be likely suffering from or be likely susceptible to ESCA comprises at least three markers, selected from the group consisting of: a CYP2S1 polypeptide, a FERMT1 polypeptide, and a ULBP2 polypeptide; or a CYP2S1 polypeptide, a ILDR1 polypeptide, and a ULBP2 polypeptide; or a CDH3 polypeptide, a CYP2S1 polypeptide, and a ILDR1 polypeptide; or a CDH3 polypeptide, a CYP2S1 polypeptide, and a EPCAM polypeptide; or a CDH3 polypeptide, a CEACAM5 polypeptide, and a TMEM238 polypeptide; or a CYP2S1 polypeptide, a LAMC2 polypeptide, and a ULBP2 polypeptide; or a CYP2S1 polypeptide, a LAMB3 polypeptide, and a ULBP2 polypeptide; or a CDH3 polypeptide, a CEACAM6 polypeptide, and a CYP2S1 polypeptide; or a CDH3 polypeptide, a CEACAM5 polypeptide, and a LAMC2 polypeptide; or a CDH3 polypeptide, a CYP2S1 polypeptide, and a LAMC2 polypeptide; or a CDH3 polypeptide, a CEACAM5 polypeptide, and a CYP2S1 polypeptide; or a CYP2S1 polypeptide, a ILDR1 polypeptide, and a LAMC2 polypeptide; or a CYP2S1 polypeptide, a FERMT1 polypeptide, and a MET polypeptide; or a HS6ST2 polypeptide, a LAMC2 polypeptide, and a RACGAP1 polypeptide; or a HS6ST2 polypeptide, a LAMB3 polypeptide, and a LAMC2 polypeptide; or combinations thereof. In some embodiments, any two markers of the 3-biomarker combinations as described herein for detection of ESCA can be used as a 2-marker combination for detection of ESCA.

In some embodiments, at least one or more marker combinations that are suitable for detection of Glioblastoma multiforme (GBM) can be included in cancer detection. In some embodiments, marker combinations can enrich a population for subjects who may likely be suffering from or be susceptible to GBM.

In some embodiments, a marker combination suitable for enriching a population for subjects who may be likely suffering from or be likely susceptible to GBM comprises at least three markers, selected from the group consisting of: a CDH2 polypeptide, a LRRN1 polypeptide, and a PRAF2 polypeptide; or a CDH2 polypeptide, a CLN5 polypeptide, and a LRRN1 polypeptide; or a CDH2 polypeptide, a LRRN1 polypeptide, and a SLC4A4 polypeptide; or a CDH2 polypeptide, a LRRN1 polypeptide, and a TMEM132A polypeptide; or a CDH2 polypeptide, a LMNB1 polypeptide, and a LRRN1 polypeptide; or a CDH2 polypeptide, a LRRN1 polypeptide, and a RAC3 polypeptide; or a CDH2 polypeptide, a GPRIN1 polypeptide, and a LRRN1 polypeptide; or a CDH2 polypeptide, a LRRN1 polypeptide, and a TMEM9 polypeptide; or a CDH2 polypeptide, a CLN5 polypeptide, and a GNG4 polypeptide; or a CDH2 polypeptide, a LRRN1 polypeptide, and a RACGAP1 polypeptide; or a CDH2 polypeptide, a LRRN1 polypeptide, and a PODXL2 polypeptide; or a CDH2 polypeptide, a IGSF3 polypeptide, and a LRRN1 polypeptide; or a CDH2 polypeptide, a HACD3 polypeptide, and a LRRN1 polypeptide; or a CDH2 polypeptide, a EPHB2 polypeptide, and a LRRN1 polypeptide; or a CDH2 polypeptide, a GOLM1 polypeptide, and a LRRN1 polypeptide; or combinations thereof. In some embodiments, any two markers of the 3-biomarker combinations as described herein for detection of GBM can be used as a 2-marker combination for detection of GBM.

In some embodiments, at least one or more marker combinations that are suitable for detection of Head and Neck squamous cell carcinoma (HNSC) can be included in cancer detection. In some embodiments, marker combinations can enrich a population for subjects who may likely be suffering from or be susceptible to HNSC.

In some embodiments, a marker combination suitable for enriching a population for subjects who may be likely suffering from or be likely susceptible to HNSC comprises at least three markers, selected from the group consisting of: a CYP2S1 polypeptide, a LAMC2 polypeptide, and a ULBP2 polypeptide; or a LAMB3 polypeptide, a LAMC2 polypeptide, and a ULBP2 polypeptide; or a LAMB3 polypeptide, a LAMC2 polypeptide, and a RAP2B polypeptide; or a CDH3 polypeptide, a CYP2S1 polypeptide, and a LAMC2 polypeptide; or a LAMC2 polypeptide, a LSR polypeptide, and a ULBP2 polypeptide; or a GALNT14 polypeptide, a LAMC2 polypeptide, and a ULBP2 polypeptide; or a HS6ST2 polypeptide, a LAMC2 polypeptide, and a ULBP2 polypeptide; or a GALNT14 polypeptide, a LAMB3 polypeptide, and a LAMC2 polypeptide; or a CYP2S1 polypeptide, a LAMB3 polypeptide, and a ULBP2 polypeptide; or a HS6ST2 polypeptide, a LAMB3 polypeptide, and a LAMC2 polypeptide; or a HS6ST2 polypeptide, a LAMC2 polypeptide, and a RAP2B polypeptide; or a CDH3 polypeptide, a EPHB2 polypeptide, and a LAMC2 polypeptide; or a CDH3 polypeptide, a HS6ST2 polypeptide, and a LAMC2 polypeptide; or a CDH3 polypeptide, a DSG2 polypeptide, and a LAMC2 polypeptide; or a HS6ST2 polypeptide, a LAMB3 polypeptide, and a ULBP2 polypeptide; or combinations thereof. In some embodiments, any two markers of the 3-biomarker combinations as described herein for detection of HNSC can be used as a 2-marker combination for detection of HNSC.

In some embodiments, at least one or more marker combinations that are suitable for detection of Kidney Chromophobe (KICH) can be included in cancer detection. In some embodiments, marker combinations can enrich a population for subjects who may likely be suffering from or be susceptible to KICH.

In some embodiments, a marker combination suitable for enriching a population for subjects who may be likely suffering from or be likely susceptible to KICH comprises at least three markers, selected from the group consisting of: a BMPR1B polypeptide, a DSG2 polypeptide, and a ILDR1 polypeptide; or a APOO polypeptide, a BMPR1B polypeptide, and a ILDR1 polypeptide; or a BMPR1B polypeptide, a HACD3 polypeptide, and a ILDR1 polypeptide; or a BMPR1B polypeptide, a CLN5 polypeptide, and a ILDR1 polypeptide; or a BMPR1B polypeptide, a GOLM1 polypeptide, and a ILDR1 polypeptide; or a AP1M2 polypeptide, a BMPR1B polypeptide, and a DSG2 polypeptide; or a CLN5 polypeptide, a PARD6B polypeptide, and a SYT13 polypeptide; or a BMPR1B polypeptide, a CDH1 polypeptide, and a ILDR1 polypeptide; or a BMPR1B polypeptide, a GPR160 polypeptide, and a ILDR1 polypeptide; or a APOO polypeptide, a BMPR1B polypeptide, and a CDH1 polypeptide; or a ALDH18A1 polypeptide, a BMPR1B polypeptide, and a SYT13 polypeptide; or a BMPR1B polypeptide, a CADM4 polypeptide, and a ILDR1 polypeptide; or a BMPR1B polypeptide, a GNPNAT1 polypeptide, and a ILDR1 polypeptide; or a AP1M2 polypeptide, a APOO polypeptide, and a BMPR1B polypeptide; or a BMPR1B polypeptide, a CDH1 polypeptide, and a GOLM1 polypeptide; or combinations thereof. In some embodiments, any two markers of the 3-biomarker combinations as described herein for detection of KICH can be used as a 2-marker combination for detection of KICH.

In some embodiments, at least one or more marker combinations that are suitable for detection of Kidney renal clear cell carcinoma (KIRC) can be included in cancer detection. In some embodiments, marker combinations can enrich a population for subjects who may likely be suffering from or be susceptible to KIRC.

In some embodiments, a marker combination suitable for enriching a population for subjects who may be likely suffering from or be likely susceptible to KIRC comprises at least three markers, selected from the group consisting of: a CLN5 polypeptide, a GALNT14 polypeptide, and a RNF128 polypeptide; or a CDH2 polypeptide, a CLN5 polypeptide, and a GALNT14 polypeptide; or a GALNT14 polypeptide, a MET polypeptide, and a RNF128 polypeptide; or a GALNT14 polypeptide, a PMEPA1 polypeptide, and a RNF128 polypeptide; or a GALNT14 polypeptide, a RAP2B polypeptide, and a RNF128 polypeptide; or a CDH2 polypeptide, a GALNT14 polypeptide, and a PMEPA1 polypeptide; or a CD24 polypeptide, a CDH2 polypeptide, and a GALNT14 polypeptide; or a CDH2 polypeptide, a DSG2 polypeptide, and a GALNT14 polypeptide; or a CDH2 polypeptide, a GALNT14 polypeptide, and a UNC13B polypeptide; or a CDH2 polypeptide, a FOLR1 polypeptide, and a GALNT14 polypeptide; or a GALNT14 polypeptide, a PMEPA1 polypeptide, and a SYT13 polypeptide; or a GALNT14 polypeptide, a RAP2B polypeptide, and a SYT13 polypeptide; or a CD24 polypeptide, a CDH2 polypeptide, and a CLN5 polypeptide; or a CLN5 polypeptide, a GALNT14 polypeptide, and a SYT13 polypeptide; or a ARFGEF3 polypeptide, a CDH2 polypeptide, and a GALNT14 polypeptide; or combinations thereof. In some embodiments, any two markers of the 3-biomarker combinations as described herein for detection of KIRC can be used as a 2-marker combination for detection of KIRC.

In some embodiments, at least one or more marker combinations that are suitable for detection of Kidney renal papillary cell carcinoma (KIRP) can be included in cancer detection. In some embodiments, marker combinations can enrich a population for subjects who may likely be suffering from or be susceptible to KIRP.

In some embodiments, a marker combination suitable for enriching a population for subjects who may be likely suffering from or be likely susceptible to KIRP comprises at least three markers, selected from the group consisting of: a CDH2 polypeptide, a CLDN4 polypeptide, and a MET polypeptide; or a GALNT14 polypeptide, a MET polypeptide, and a RNF128 polypeptide; or a CD24 polypeptide, a CDH2 polypeptide, and a GALNT14 polypeptide; or a CD24 polypeptide, a CDH2 polypeptide, and a MET polypeptide; or a CLN5 polypeptide, a GALNT14 polypeptide, and a RNF128 polypeptide; or a CDH2 polypeptide, a CLN5 polypeptide, and a GALNT14 polypeptide; or a GALNT14 polypeptide, a RAP2B polypeptide, and a SYT13 polypeptide; or a CD24 polypeptide, a CDH2 polypeptide, and a CLN5 polypeptide; or a CDH2 polypeptide, a ILDR1 polypeptide, and a RAP2B polypeptide; or a CDH2 polypeptide, a GALNT14 polypeptide, and a UNC13B polypeptide; or a CDH2 polypeptide, a DSG2 polypeptide, and a GALNT14 polypeptide; or a CDH2 polypeptide, a CLN5 polypeptide, and a ILDR1 polypeptide; or a CDH2 polypeptide, a ILDR1 polypeptide, and a SMPDL3B polypeptide; or a CD24 polypeptide, a CDH2 polypeptide, and a UNC13B polypeptide; or a CDH2 polypeptide, a CLDN4 polypeptide, and a CLN5 polypeptide; or combinations thereof. In some embodiments, any two markers of the 3-biomarker combinations as described herein for detection of KIRP can be used as a 2-marker combination for detection of KIRP.

In some embodiments, at least one or more marker combinations that are suitable for detection of Liver hepatocellular carcinoma (LIHC) can be included in cancer detection. In some embodiments, marker combinations can enrich a population for subjects who may likely be suffering from or be susceptible to LIHC.

In some embodiments, one or more markers that are suitable for detection of LIHC and are useful to be included in cancer detection can be selected from: (i) polypeptides encoded by human genes as follows: ACBD3, ACSL4, ACY3, ANXA13, AP1M2, APOO, ATP1B1, ATP2B2, ATRN, CADM1, CAP2, CD63, CDH2, CDHR5, CKAP4, CLGN, COX6C, CXADR, CYP4F11, EPCAM, EPHX1, FGFR4, G6PD, GBA, GJB1, GLUL, GPC3, HKDC1, HPN, HSD17B2, IGSF8, KDELR1, LAD1, LAMC1, LAMTOR2, LBR, LSR, MARCKS, MARVELD2, MET, MPC2, MUC13, NAT8, NDUFA2, OCLN, PDZK1, PIGT, QPCTL, RAC3, RALBP1, ROBO1, ROMO1, S100P, SCAMP3, SCGN, SDC2, SLC22A9, SLC29A1, SLC2A2, SLC35B2, SLC38A3, TFR2, TM4SF4, TMCO1, TMEM209, TMPRSS6, TOMM20, TOMM22, TOR1A1P2, UGT1A6, UGT1A9, UGT2B7, UNC13B, VAT1, VPS28, DKK1, DLK1, ENPP3, MUC1, PI4K2A, PLVAP, SPINK1, TNFRSF10A, TNFSF18, and combinations thereof; and/or (ii) carbohydrate-dependent markers as follows: Lewis Y antigen (also known as CD174), Tn antigen, Thomsen-Friedenreich (T, TF) antigen, Sialyl Lewis X (sLex) antigen (also known as Sialyl SSEA-1 (SLX)), and combinations thereof.

In some embodiments, one or more markers that are suitable for detection of LIHC and are useful to be included in cancer detection can be selected from: (i) polypeptides encoded by human genes as follows: ACSL4, ANXA13, AP1M2, ATP1B1, CAP2, CDH2, CDHR5, CKAP4, EPCAM, GBA, GJB1, GLUL, GPC3, MARVELD2, MET, MUC13, NAT8, PDZK1, ROBO1, SCGN, SLC22A9, SLC2A2, SLC35B2, SLC38A3, TFR2, TM4SF4, TMPRSS6, TOMM20, UGT1A9, UGT2B7, and combinations thereof; and/or (ii) carbohydrate-dependent markers as follows: Lewis Y antigen (also known as CD174), Sialyl Lewis X (sLex) antigen (also known as Sialyl SSEA-1 (SLX)), T antigen, Tn antigen, and combinations thereof.

In some embodiments, a marker combination suitable for enriching a population for subjects who may be likely suffering from or be likely susceptible to LIHC comprises at least three markers, selected from the group consisting of: a APOO polypeptide, a GJB1 polypeptide, and a IGSF3 polypeptide; or a CDH1 polypeptide, a CDH2 polypeptide, and a HS6ST2 polypeptide; or a CDH1 polypeptide, a CDH2 polypeptide, and a ILDR1 polypeptide; or a GJB1 polypeptide, a IGSF3 polypeptide, and a KPNA2 polypeptide; or a GJB1 polypeptide, a IGSF3 polypeptide, and a RAP2B polypeptide; or a CDH2 polypeptide, a ILDR1 polypeptide, and a RAP2B polypeptide; or a CDH2 polypeptide, a ILDR1 polypeptide, and a MAL2 polypeptide; or a CDH2 polypeptide, a CLN5 polypeptide, and a ILDR1 polypeptide; or a GJB1 polypeptide, a IGSF3 polypeptide, and a LAPTM4B polypeptide; or a CDH2 polypeptide, a CLN5 polypeptide, and a SYT13 polypeptide; or a CDH2 polypeptide, a CLN5 polypeptide, and a EPCAM polypeptide; or a CDH1 polypeptide, a CDH2 polypeptide, and a MARCKSL1 polypeptide; or a CDH2 polypeptide, a ILDR1 polypeptide, and a UNC13B polypeptide; or a CDH2 polypeptide, a FAM241B polypeptide, and a ILDR1 polypeptide; or a GJB1 polypeptide, a IGSF3 polypeptide, and a RCC2 polypeptide; or combinations thereof. In some embodiments, any two markers of the 3-biomarker combinations as described herein for detection of LIHC can be used as a 2-marker combination for detection of LIHC.

In some embodiments, at least one or more marker combinations that are suitable for detection of lung cancer can be included in cancer detection. In some embodiments, provided marker combinations can enrich a population for subjects who may likely be suffering from or be susceptible to lung cancer.

In some embodiments, one or more markers that are suitable for detection of lung cancer and are useful to be included in cancer detection can be selected from: (i) polypeptides encoded by human genes as follows: ADGRF1, ABCC3, ALCAM, ARSL, B3GNT3, B3GNT5, CDCP1, CDH1, CDH3, CD55, CD274 (PD-L1), CEACAM5, CEACAM6, CELSR1, CLDN18, CLDN3, CLDN4, CLDN7, CLIC6, DMBT1, DSG2, EGFR, EPCAM, EPHX3, EVA1A, FAM241B, FOLR1, FXYD3, GALNT14, GJB1, GJB2, GPC4, HAS3, HS6ST2, IG1FR, KDELR3, KRTCAP3, LAMB3, LAPTM4B, LARGE2, LFNG, LSR, MAL2, MANEAL, MET, MSLN, MUC1, MUC21, NRCAM, PIGT, PODXL2, PRRG4, PRSS21, ROS1, SDC1, SERINC2, SEZ6L2, SLC34A2, SLC44A4, SLC6A14, SLC7A7, SLC7A11, SMIM22, SMPDL3B, ST14, TACSTD2, TMC4, TMC5, TMEM45B, TMPRSS2, TMPRSS4, TNFRSF10B, TSPAN1, TSPAN8, UCHL1, and combinations thereof; and/or (ii) carbohydrate-dependent markers as follows: Lewis X antigen, Lewis Y antigen (also known as CD174), SialylTn (sTn) antigen, Sialyl Lewis X (sLex) antigen (also known as Sialyl SSEA-1 (SLX)), T antigen, Tn antigen, and combinations thereof.

In some embodiments, one or more markers that are suitable for detection of lung cancer and are useful to be included in cancer detection can be selected from: (i) polypeptides encoded by human genes as follows: ADGRF1, ALCAM, B3GNT3, B3GNT5, CDCP1, CDH1, CDH3, CD55, CD274 (PD-L1), CEACAM5, CEACAM6, CLDN3, CLDN4, DSG2, EGFR, EPCAM, FAM241B, FOLR1, FXYD3, GALNT14, GJB1, GJB2, HAS3, IG1FR, LAMB3, LAPTM4B, LARGE2, MAL2, MET, MSLN, MUC1, NRCAM, PIGT, PODXL2, PRSS21, ROS1, SDC1, SLC34A2, SLC7A11, SMIM22, SMPDL3B, ST14, UCHL1, TACSTD2, TMPRSS4, TSPAN8, TNFRSF10B, and combinations thereof; and/or (ii) carbohydrate-dependent markers as follows: Lewis X antigen, Lewis Y antigen (also known as CD174), SialylTn (sTn) antigen, Sialyl Lewis X (sLex) antigen (also known as Sialyl SSEA-1 (SLX)), T antigen, Tn antigen, and combinations thereof.

In some embodiments, at least one or more marker combinations that are suitable for detection of Lung adenocarcinoma (LUAD)
can be included in cancer detection. In some embodiments, marker combinations can enrich a population for subjects who may likely be suffering from or be susceptible to LUAD.

In some embodiments, a marker combination suitable for enriching a population for subjects who may be likely suffering from or be likely susceptible to LUAD comprises at least three markers, selected from the group consisting of: a CEACAM6 polypeptide, a HS6ST2 polypeptide, and a PODXL2 polypeptide; or a CEACAM6 polypeptide, a HS6ST2 polypeptide, and a LARGE2 polypeptide; or a CEACAM6 polypeptide, a HS6ST2 polypeptide, and a MARCKSL1 polypeptide; or a HS6ST2 polypeptide, a MAL2 polypeptide, and a SMPDL3B polypeptide; or a CEACAM6 polypeptide, a HS6ST2 polypeptide, and a LSR polypeptide; or a CEACAM6 polypeptide, a HS6ST2 polypeptide, and a RAP2B polypeptide; or a AP1M2 polypeptide, a CEACAM6 polypeptide, and a HS6ST2 polypeptide; or a APOO polypeptide, a CEACAM6 polypeptide, and a HS6ST2 polypeptide; or a ALDH18A1 polypeptide, a CEACAM6 polypeptide, and a HS6ST2 polypeptide; or a CDH3 polypeptide, a CEACAM6 polypeptide, and a HS6ST2 polypeptide; or a CEACAM6 polypeptide, a HS6ST2 polypeptide, and a NUP210 polypeptide; or a CEACAM6 polypeptide, a GPR160 polypeptide, and a HS6ST2 polypeptide; or a CEACAM6 polypeptide, a HS6ST2 polypeptide, and a RCC2 polypeptide; or a CEACAM6 polypeptide, a HS6ST2 polypeptide, and a LAMC2 polypeptide; or a HS6ST2 polypeptide, a LAMC2 polypeptide, and a SMPDL3B polypeptide; or combinations thereof. In some embodiments, any two markers of the 3-biomarker combinations as described herein for detection of LUAD can be used as a 2-marker combination for detection of LUAD.

In some embodiments, at least one or more marker combinations that are suitable for detection of Lung squamous cell carcinoma (LUSC) can be included in cancer detection. In some embodiments, marker combinations can enrich a population for subjects who may likely be suffering from or be susceptible to LUSC.

In some embodiments, a marker combination suitable for enriching a population for subjects who may be likely suffering from or be likely susceptible to LUSC comprises at least three markers, selected from the group consisting of: a CYP2S1 polypeptide, a HS6ST2 polypeptide, and a LAMC2 polypeptide; or a CYP2S1 polypeptide, a HS6ST2 polypeptide, and a KPNA2 polypeptide; or a APOO polypeptide, a CYP2S1 polypeptide, and a HS6ST2 polypeptide; or a CYP2S1 polypeptide, a FAM241B polypeptide, and a HS6ST2 polypeptide; or a CYP2S1 polypeptide, a HS6ST2 polypeptide, and a LSR polypeptide; or a CYP2S1 polypeptide, a ILDR1 polypeptide, and a ULBP2 polypeptide; or a CDH3 polypeptide, a CYP2S1 polypeptide, and a ILDR1 polypeptide; or a CYP2S1 polypeptide, a HS6ST2 polypeptide, and a RCC2 polypeptide; or a HS6ST2 polypeptide, a LAMC2 polypeptide, and a RAP2B polypeptide; or a CYP2S1 polypeptide, a LAMC2 polypeptide, and a ULBP2 polypeptide; or a CYP2S1 polypeptide, a HS6ST2 polypeptide, and a LAMB3 polypeptide; or a CYP2S1 polypeptide, a HS6ST2 polypeptide, and a RACGAP1 polypeptide; or a CYP2S1 polypeptide, a LAMB3 polypeptide, and a ULBP2 polypeptide; or a CYP2S1 polypeptide, a HS6ST2 polypeptide, and a LAPTM4B polypeptide; or a HS6ST2 polypeptide, a LAMC2 polypeptide, and a LSR polypeptide; or combinations thereof. In some embodiments, any two markers of the 3-biomarker combinations as described herein for detection of LUSC can be used as a 2-marker combination for detection of LUSC.

In some embodiments, at least one or more marker combinations that are suitable for detection of Mesothelioma (MESO) can be included in cancer detection. In some embodiments, marker combinations can enrich a population for subjects who may likely be suffering from or be susceptible to MESO.

In some embodiments, a marker combination suitable for enriching a population for subjects who may be likely suffering from or be likely susceptible to MESO comprises at least three markers, selected from the group consisting of: a CDH2 polypeptide, a EPHB2 polypeptide, and a LRRN1 polypeptide; or a CDH1 polypeptide, a CDH2 polypeptide, and a CDH3 polypeptide; or a CDH2 polypeptide, a LAMC2 polypeptide, and a SMPDL3B polypeptide; or a CDH2 polypeptide, a CDH3 polypeptide, and a LAMB3 polypeptide; or a CDH1 polypeptide, a CDH2 polypeptide, and a LAMC2 polypeptide; or a CDH2 polypeptide, a LAPTM4B polypeptide, and a SMPDL3B polypeptide; or a CDH2 polypeptide, a LMNB1 polypeptide, and a LRRN1 polypeptide; or a CDH2 polypeptide, a LRRN1 polypeptide, and a TMEM132A polypeptide; or a AP1M2 polypeptide, a CDH2 polypeptide, and a CDH3 polypeptide; or a CDH3 polypeptide, a EPHB2 polypeptide, and a LAMC2 polypeptide; or a CDH2 polypeptide, a CDH3 polypeptide, and a SHISA2 polypeptide; or a AP1M2 polypeptide, a CDH2 polypeptide, and a SLC39A6 polypeptide; or a CDH2 polypeptide, a LRRN1 polypeptide, and a RCC2 polypeptide; or a AP1M2 polypeptide, a CDH2 polypeptide, and a CLN5 polypeptide; or a CDH2 polypeptide, a LRRN1 polypeptide, and a RACGAP1 polypeptide; or combinations thereof. In some embodiments, any two markers of the 3-biomarker combinations as described herein for detection of MESO can be used as a 2-marker combination for detection of MESO.

In some embodiments, at least one or more marker combinations that are suitable for detection of Ovarian serous cystadenocarcinoma (OV) can be included in cancer detection. In some embodiments, marker combinations can enrich a population for subjects who may likely be suffering from or be susceptible to OV.

In some embodiments, one or more markers that are suitable for detection of OV and are useful to be included in cancer detection can be selected from: (i) polypeptides encoded by human genes as follows: ALPL, AQP5, BCAM, BST2, CD24, CD74, CDH6, CHODL, CLDN16, CLDN3, CLDN6, CXCR4, DDR1, EFNB1, EPCAM, FOLR1, HTR3A, LEMD1, LRRTM1, LY6E, MSLN, MUC1, MUC16, NOTCH3, PLXNB1, PTGS1, SLC2A1, SLC34A2, SPINT2, ST14, TACSTD2, TNFRSF12A, and combinations thereof; and/or (ii) carbohydrate-dependent markers as follows: Lewis Y antigen (also known as CD174), SialylTn (sTn) antigen, Sialyl Lewis A antigen (also known as CA19-9), Sialyl Lewis X (sLex) antigen (also known as Sialyl SSEA-1 (SLX)), T antigen, Tn antigen, and combinations thereof.

In some embodiments, a marker combination suitable for enriching a population for subjects who may be likely suffering from or be likely susceptible to OV comprises at least three markers, selected from the group consisting of: a CLDN3 polypeptide, a EPHB2 polypeptide, and a FOLR1 polypeptide; or a CLDN3 polypeptide, a FOLR1 polypeptide, and a LAPTM4B polypeptide; or a CLDN3 polypeptide, a FOLR1 polypeptide, and a MARCKSL1 polypeptide; or a CLDN3 polypeptide, a FZD2 polypeptide, and a LAPTM4B polypeptide; or a CDH2 polypeptide, a CLDN3 polypeptide, and a LAPTM4B polypeptide; or a FOLR1 polypeptide, a KPNA2 polypeptide, and a VTCN1 polypeptide; or a BMPR1B polypeptide, a CLDN3 polypeptide, and a TMEM238 polypeptide; or a FOLR1 polypeptide, a MARCKSL1 polypeptide, and a SMPDL3B polypeptide; or a FOLR1 polypeptide, a LMNB1 polypeptide, and a VTCN1 polypeptide; or a CLDN3 polypeptide, a FZD2 polypeptide, and a VTCN1 polypeptide; or a CDH2 polypeptide, a FOLR1 polypeptide, and a SMPDL3B polypeptide; or a BMPR1B polypeptide, a CLDN3 polypeptide, and a LAPTM4B polypeptide; or a CDH2 polypeptide, a CLDN3 polypeptide, and a FZD2 polypeptide; or a APOO polypeptide, a CLDN3 polypeptide, and a FZD2 polypeptide; or a CDH2 polypeptide, a CLDN3 polypeptide, and a RCC2 polypeptide; or combinations thereof. In some embodiments, any two markers of the 3-biomarker combinations as described herein for detection of OV can be used as a 2-marker combination for detection of OV.

In some embodiments, at least one or more marker combinations that are suitable for detection of Pancreatic adenocarcinoma (PAAD) can be included in cancer detection. In some embodiments, marker combinations can enrich a population for subjects who may likely be suffering from or be susceptible to PAAD.

In some embodiments, one or more markers that are suitable for detection of PAAD and are useful to be included in cancer detection can be selected from: (i) polypeptides encoded by human genes as follows: ADGRG1, ANO1, AP1M2, ATP1B1, CARD11, CDCP1, CDH1, CDH11, CDH17, CEACAM5, CEACAM6, CFTR, CLIC3, CLN5, CNTN1, CYP2S1, DSG2, EPCAM, EPHA2, FERIL6, FERMT1, GALNT3, GALNT5, GATM, GCNT3, GOLM1, GP2, GPRC5A, GPX8, HACD3, HKDC1, HSD17B2, ITGA11, ITGA2, ITGB4, ITGB6, LAD1, LAMA3, LAMB3, LAMC2, LOXL2, LSR, MARCKSL1, MET, MMP14, MOXD1, MSLN, MUC1, MUC13, PCDH1, PIGT, PIK3AP1, PROM1, QSOX1, RAB25, RAB27B, RAP2B, S100A6, S100P, SCGN, SDR16C5, SHROOM3, SLC4A4, SMPDL3B, SPARC, SRC, ST14, TACSTD2, TESC, THY1, TJP3, TSPAN8, VASP, VNN1, VWA1, ADAM17, BAG3, CCN2, CETN1, EGFR, ERBB3, GUCY2C, ICAM1, IGF1R, IL1A, MDM2, MUC17, MUC5AC, MUCL1, NOTCH2, NOTCH3, PLAUR, SLC44A4, TF, TFRC, TNFRSF10B, and combinations thereof; and/or (ii) carbohydrate-dependent markers as follows: Lewis Y antigen (also known as CD174), Sialyl Lewis A antigen (also known as CA19-9), SialylTn (sTn) antigen, Sialyl Lewis X (sLex) antigen (also known as Sialyl SSEA-1 (SLX)), T antigen, Tn antigen, and combinations thereof.

In some embodiments, one or more markers that are suitable for detection of PAAD and are useful to be included in cancer detection can be selected from: (i) polypeptides encoded by human genes as follows: AP1M2, CARD11, CDH1, CEACAM5, CEACAM6, CFTR, CLN5, CYP2S1, EPCAM, FER1L6, FERMT1, GALNT3, GALNT5, GCNT3, HSD17B2, ITGB6, LAD1, LAMB3, LAMC2, LSR, MARCKSL1, MSLN, MUC1, MUC13, RAB25, S100P, SCGN, SLC4A4, TACSTD2, TESC, and combinations thereof; and/or (ii) carbohydrate-dependent markers as follows: Lewis Y antigen (also known as CD174), SialylTn (sTn) antigen, Sialyl Lewis X (sLex) antigen (also known as Sialyl SSEA-1 (SLX)), T antigen, Tn antigen, and combinations thereof.

In some embodiments, a marker combination suitable for enriching a population for subjects who may be likely suffering from or be likely susceptible to PAAD comprises at least three markers, selected from the group consisting of: a B3GNT3 polypeptide, a LAMC2 polypeptide, and a PMEPA1 polypeptide; or a B3GNT3 polypeptide, a LAMC2 polypeptide, and a SHISA2 polypeptide; or a B3GNT3 polypeptide, a FZD2 polypeptide, and a LAMC2 polypeptide; or a CYP2S1 polypeptide, a SYT13 polypeptide, and a VTCN1 polypeptide; or a B3GNT3 polypeptide, a LAMC2 polypeptide, and a MET polypeptide; or a CDH3 polypeptide, a CYP2S1 polypeptide, and a SYT13 polypeptide; or a CDH3 polypeptide, a CEACAM6 polypeptide, and a GJB1 polypeptide; or a CDH3 polypeptide, a FZD2 polypeptide, and a SYT13 polypeptide; or a ILDR1 polypeptide, a LAMB3 polypeptide, and a LAMC2 polypeptide; or a CEACAM5 polypeptide, a PMEPA1 polypeptide, and a SHISA2 polypeptide; or a PARD6B polypeptide, a PMEPA1 polypeptide, and a SYT13 polypeptide; or a CYP2S1 polypeptide, a ILDR1 polypeptide, and a PMEPA1 polypeptide; or a CDH3 polypeptide, a CEACAM5 polypeptide, and a PMEPA1 polypeptide; or a CDH3 polypeptide, a SMPDL3B polypeptide, and a SYT13 polypeptide; or a GALNT14 polypeptide, a PMEPA1 polypeptide, and a SYT13 polypeptide; or combinations thereof. In some embodiments, any two markers of the 3-biomarker combinations as described herein for detection of PAAD can be used as a 2-marker combination for detection of PAAD.

In some embodiments, at least one or more marker combinations that are suitable for detection of Pheochromocytoma and Paraganglioma (PCPG) can be included in cancer detection. In some embodiments, marker combinations can enrich a population for subjects who may likely be suffering from or be susceptible to PCPG.

In some embodiments, a marker combination suitable for enriching a population for subjects who may be likely suffering from or be likely susceptible to PCPG comprises at least three markers, selected from the group consisting of: a CDH2 polypeptide, a CLN5 polypeptide, and a GNG4 polypeptide; or a BMPR1B polypeptide, a CDH2 polypeptide, and a GNG4 polypeptide; or a BMPR1B polypeptide, a CDH2 polypeptide, and a HS6ST2 polypeptide; or a BMPR1B polypeptide, a CLGN polypeptide, and a PODXL2 polypeptide; or a ARFGEF3 polypeptide, a CDH2 polypeptide, and a GALNT14 polypeptide; or a BMPR1B polypeptide, a CD24 polypeptide, and a GPRIN1 polypeptide; or a CLGN polypeptide, a PODXL2 polypeptide, and a SLC39A6 polypeptide; or a CDH2 polypeptide, a GALNT14 polypeptide, and a PODXL2 polypeptide; or a BMPR1B polypeptide, a CDH2 polypeptide, and a PODXL2 polypeptide; or a APOO polypeptide, a CDH2 polypeptide, and a PODXL2 polypeptide; or a CDH2 polypeptide, a PODXL2 polypeptide, and a UNC13B polypeptide; or a CDH2 polypeptide, a CLN5 polypeptide, and a PODXL2 polypeptide; or a CD24 polypeptide, a CDH2 polypeptide, and a PRAF2 polypeptide; or a CD24 polypeptide, a CDH2 polypeptide, and a SLC39A6 polypeptide; or a BMPR1B polypeptide, a ELAPOR1 polypeptide, and a GPRIN1 polypeptide; or combinations thereof. In some embodiments, any two markers of the 3-biomarker combinations as described herein for detection of PCPG can be used as a 2-marker combination for detection of PCPG.

In some embodiments, at least one or more marker combinations that are suitable for detection of Prostate adenocarcinoma (PRAD) can be included in cancer detection. In some embodiments, marker combinations can enrich a population for subjects who may likely be suffering from or be susceptible to PRAD.

In some embodiments, one or more markers that are suitable for detection of PRAD and are useful to be included in cancer detection can be selected from: (i) polypeptides encoded by human genes as follows: ABCC4, ABHD17C, ADI1, AGTRAP, AP1M2, APOO, ARFGEF3, ATP2C1, BCAM, CADM4, CANT1, CDH1, CHMP4C, CLDN3, CLDN4, CLGN, CLN5, CYB561, DNAJC30, ENPP5, EPCAM, ERGIC1, FAAH, FOLH1, GALNT3, GNG4, GNPNAT1, GOLM1, GRHL2, HID1, HOMER2, HPN, LCP1, LRIG1, MAP7, MARCKSL1, MARVELD2, MBOAT2, MIA3, MUC1, NAAA, NDUFA2, PMEPA1, PODXL2, PPP3CA, PRSS8, RAB3B, RAB3D, RAP1GAP, RDH11, SCARB2, SERINC5, SFXN2, SHROOM2, SHROOM3, SLC35F2, SLC39A6, SLC39A7, SLC4A4, SMPDL3B, SORD, STEAP1, STEAP2, SYNGR2, SYT7, TMC5, TMED3, TMEM141, TMEM192, TMEM9, TMPRSS2, TRPM4, TSPAN1, UNC13B, VWA1, YIPF1, ADAM17, CCL2, CD274, CD38, CLEC2D, ERBB2, FLNA, FLNB, GPC1, IL6, ITGAV, KLK3, KLKB1, PLAC1, PPPIR3A, PSCA, PVR, SLC44A4, TGFBR2, TNFRSF4, TNFSF11, VEGFC, and combinations thereof; and/or (ii) carbohydrate-dependent markers as follows: Tn antigen, SialylTn (sTn) antigen, Thomsen-Friedenreich (T, TF) antigen, Lewis Y antigen (also known as CD174), Sialyl Lewis X (sLex) antigen (also known as Sialyl SSEA-1 (SLX)), and combinations thereof.

In some embodiments, one or more markers that are suitable for detection of PRAD and are useful to be included in cancer detection can be selected from: (i) polypeptides encoded by human genes as follows: ABCC4, AP1M2, ARFGEF3, CANT1, CD38, CDH1, CLDN3, CLDN4, CLGN, ENPP5, FOLH1, GOLM1, GRHL2, MAP7, MARCKSL1, MUC1, PMEPA1, PODXL2, PPP3CA, PSCA, RAB3B, RAB3D, RDH11, SLC39A6, SLC4A4, SMPDL3B, SORD, STEAP1, STEAP2, SYT7, TMPRSS2, TRPM4, TSPAN1, UNC13B, and combinations thereof; and/or (ii) carbohydrate-dependent markers as follows: Lewis Y antigen (also known as CD174), SialylTn (sTn) antigen, Sialyl Lewis X (sLex) antigen (also known as Sialyl SSEA-1 (SLX)), T antigen, Tn antigen, and combinations thereof.

In some embodiments, a marker combination suitable for enriching a population for subjects who may be likely suffering from or be likely susceptible to PRAD comprises at least three markers, selected from the group consisting of: a BMPR1B polypeptide, a CLDN3 polypeptide, and a MARCKSL1 polypeptide; or a BMPR1B polypeptide, a CLDN3 polypeptide, and a GOLM1 polypeptide; or a BMPR1B polypeptide, a CLDN3 polypeptide, and a PODXL2 polypeptide; or a PODXL2 polypeptide, a SLC39A6 polypeptide, and a SLC44A4 polypeptide; or a AP1M2 polypeptide, a BMPR1B polypeptide, and a PODXL2 polypeptide; or a BMPR1B polypeptide, a ILDR1 polypeptide, and a PODXL2 polypeptide; or a BMPR1B polypeptide, a CLDN4 polypeptide, and a PODXL2 polypeptide; or a BMPR1B polypeptide, a ILDR1 polypeptide, and a MARCKSL1 polypeptide; or a BMPR1B polypeptide, a EPCAM polypeptide, and a PODXL2 polypeptide; or a AP1M2 polypeptide, a BMPR1B polypeptide, and a GOLM1 polypeptide; or a BMPR1B polypeptide, a ILDR1 polypeptide, and a SLC39A6 polypeptide; or a BMPR1B polypeptide, a CANT1 polypeptide, and a ILDR1 polypeptide; or a AP1M2 polypeptide, a BMPR1B polypeptide, and a MARCKSL1 polypeptide; or a BMPR1B polypeptide, a MARCKSL1 polypeptide, and a SLC44A4 polypeptide; or a MARCKSL1 polypeptide, a SLC39A6 polypeptide, and a SLC44A4 polypeptide; or combinations thereof. In some embodiments, any two markers of the 3-biomarker combinations as described herein for detection of PRAD can be used as a 2-marker combination for detection of PRAD.

In some embodiments, at least one or more marker combinations that are suitable for detection of Rectum adenocarcinoma (READ) can be included in cancer detection. In some embodiments, marker combinations can enrich a population for subjects who may likely be suffering from or be susceptible to READ.

In some embodiments, markers or marker combinations for READ detection that are useful to be included in cancer detection are described in U.S. Provisional Application No. 63/224,378, (the “'378 application”) and the International PCT Application that claims priority to the '378 application and was filed on Jul. 21, 2022 the entire content of each of which is incorporated herein by reference.

In some embodiments, a marker combination suitable for enriching a population for subjects who may be likely suffering from or be likely susceptible to READ comprises at least three markers, selected from the group consisting of: a CDH17 polypeptide, a CDH3 polypeptide, and a FERMT1 polypeptide; or a CDH17 polypeptide, a CDH3 polypeptide, and a GALNT6 polypeptide; or a CDH17 polypeptide, a CDH3 polypeptide, and a PMEPA1 polypeptide; or a CDH3 polypeptide, a CYP2S1 polypeptide, and a GJB1 polypeptide; or a CDH17 polypeptide, a CDH3 polypeptide, and a RNF128 polypeptide; or a CDH17 polypeptide, a CDH3 polypeptide, and a CYP2S1 polypeptide; or a CDH3 polypeptide, a CEACAM5 polypeptide, and a FERMT1 polypeptide; or a CDH3 polypeptide, a CLDN3 polypeptide, and a CYP2S1 polypeptide; or a CDH17 polypeptide, a CDH3 polypeptide, and a MARCKSL1 polypeptide; or a CDH3 polypeptide, a CYP2S1 polypeptide, and a EPCAM polypeptide; or a CDH3 polypeptide, a CEACAM5 polypeptide, and a CLN5 polypeptide; or a CDH3 polypeptide, a CEACAM5 polypeptide, and a CYP2S1 polypeptide; or a CDH3 polypeptide, a CEACAM6 polypeptide, and a EPHB2 polypeptide; or a CDH3 polypeptide, a CEACAM5 polypeptide, and a PMEPA1 polypeptide; or a CDH3 polypeptide, a CEACAM5 polypeptide, and a MARCKSL1 polypeptide; or combinations thereof. In some embodiments, any two markers of the 3-biomarker combinations as described herein for detection of READ can be used as a 2-marker combination for detection of READ.

In some embodiments, at least one or more marker combinations that are suitable for detection of Sarcoma (SARC) can be included in cancer detection. In some embodiments, marker combinations can enrich a population for subjects who may likely be suffering from or be susceptible to SARC.

In some embodiments, a marker combination suitable for enriching a population for subjects who may be likely suffering from or be likely susceptible to SARC comprises at least three markers, selected from the group consisting of: a CDH2 polypeptide, a LMNB1 polypeptide, and a LRRN1 polypeptide; or a CDH2 polypeptide, a EPHB2 polypeptide, and a LRRN1 polypeptide; or a CDH2 polypeptide, a LRRN1 polypeptide, and a RACGAP1 polypeptide; or a GNG4 polypeptide, a LMNB1 polypeptide, and a LRRN1 polypeptide; or a CDH2 polypeptide, a LRRN1 polypeptide, and a RAC3 polypeptide; or a CDH2 polypeptide, a LRRN1 polypeptide, and a PRAF2 polypeptide; or a CDH2 polypeptide, a CLN5 polypeptide, and a SHISA2 polypeptide; or a CDH2 polypeptide, a IGSF3 polypeptide, and a SHISA2 polypeptide; or a GNG4 polypeptide, a LRRN1 polypeptide, and a RACGAP1 polypeptide; or a GPRIN1 polypeptide, a LRRN1 polypeptide, and a PRAF2 polypeptide; or a CDH2 polypeptide, a IGSF3 polypeptide, and a LRRN1 polypeptide; or a CDH2 polypeptide, a LRRN1 polypeptide, and a RCC2 polypeptide; or a CDH2 polypeptide, a GPRIN1 polypeptide, and a LRRN1 polypeptide; or a CDH2 polypeptide, a CLN5 polypeptide, and a LRRN1 polypeptide; or a GOLM1 polypeptide, a GPRIN1 polypeptide, and a LRRN1 polypeptide; or combinations thereof. In some embodiments, any two markers of the 3-biomarker combinations as described herein for detection of SARC can be used as a 2-marker combination for detection of SARC.

In some embodiments, at least one or more marker combinations that are suitable for detection of Skin Cutaneous Melanoma (SKCM) can be included in cancer detection. In some embodiments, marker combinations can enrich a population for subjects who may likely be suffering from or be susceptible to SKCM.

In some embodiments, a marker combination suitable for enriching a population for subjects who may be likely suffering from or be likely susceptible to SKCM comprises at least three markers, selected from the group consisting of: a GJB1 polypeptide, a IGSF3 polypeptide, and a RAP2B polypeptide; or a GJB1 polypeptide, a IGSF3 polypeptide, and a RCC2 polypeptide; or a GJB1 polypeptide, a IGSF3 polypeptide, and a KPNA2 polypeptide; or a APOO polypeptide, a GJB1 polypeptide, and a IGSF3 polypeptide; or a GJB1 polypeptide, a IGSF3 polypeptide, and a LAPTM4B polypeptide; or a GJB1 polypeptide, a IGSF3 polypeptide, and a SLC39A6 polypeptide; or a GJB1 polypeptide, a KDELR3 polypeptide, and a SHISA2 polypeptide; or a APOO polypeptide, a CDH3 polypeptide, and a GJB1 polypeptide; or a CDH3 polypeptide, a GJB1 polypeptide, and a KPNA2 polypeptide; or a CDH3 polypeptide, a GJB1 polypeptide, and a LAPTM4B polypeptide; or a CDH3 polypeptide, a GJB1 polypeptide, and a RPN2 polypeptide; or a CDH3 polypeptide, a GJB1 polypeptide, and a RPN1 polypeptide; or a CDH3 polypeptide, a GJB1 polypeptide, and a SLC35A2 polypeptide; or a ALDH18A1 polypeptide, a CDH3 polypeptide, and a GJB1 polypeptide; or a CDH2 polypeptide, a IGSF3 polypeptide, and a SHISA2 polypeptide; or combinations thereof. In some embodiments, any two markers of the 3-biomarker combinations as described herein for detection of SKCM can be used as a 2-marker combination for detection of SKCM.

In some embodiments, at least one or more marker combinations that are suitable for detection of Stomach adenocarcinoma (STAD) can be included in cancer detection. In some embodiments, marker combinations can enrich a population for subjects who may likely be suffering from or be susceptible to STAD.

In some embodiments, a marker combination suitable for enriching a population for subjects who may be likely suffering from or be likely susceptible to STAD comprises at least three markers, selected from the group consisting of: a CDH3 polypeptide, a CYP2S1 polypeptide, and a SYT13 polypeptide; or a CYP2S1 polypeptide, a FERMT1 polypeptide, and a PMEPA1 polypeptide; or a CYP2S1 polypeptide, a ILDR1 polypeptide, and a PMEPA1 polypeptide; or a CDH3 polypeptide, a CYP2S1 polypeptide, and a GJB1 polypeptide; or a CDH17 polypeptide, a CDH3 polypeptide, and a CYP2S1 polypeptide; or a CYP2S1 polypeptide, a FERMT1 polypeptide, and a MET polypeptide; or a CDH3 polypeptide, a CEACAM5 polypeptide, and a TMEM238 polypeptide; or a MARCKSL1 polypeptide, a PODXL2 polypeptide, and a TMEM238 polypeptide; or a CDH3 polypeptide, a CLDN3 polypeptide, and a CYP2S1 polypeptide; or a CDH17 polypeptide, a CDH3 polypeptide, and a SHISA2 polypeptide; or a CDH17 polypeptide, a CDH3 polypeptide, and a GALNT6 polypeptide; or a CDH17 polypeptide, a CDH3 polypeptide, and a PMEPA1 polypeptide; or a CDH3 polypeptide, a CEACAM6 polypeptide, and a CYP2S1 polypeptide; or a CDH17 polypeptide, a CDH3 polypeptide, and a FERMT1 polypeptide; or a CDH17 polypeptide, a FOLR1 polypeptide, and a MET polypeptide; or combinations thereof. In some embodiments, any two markers of the 3-biomarker combinations as described herein for detection of STAD can be used as a 2-marker combination for detection of STAD.

In some embodiments, at least one or more marker combinations that are suitable for detection of Testicular Germ Cell Tumors (TGCT) can be included in cancer detection. In some embodiments, marker combinations can enrich a population for subjects who may likely be suffering from or be susceptible to TGCT.

In some embodiments, a marker combination suitable for enriching a population for subjects who may be likely suffering from or be likely susceptible to TGCT comprises at least three markers, selected from the group consisting of: a CDH2 polypeptide, a EPCAM polypeptide, and a RCC2 polypeptide; or a CDH2 polypeptide, a CDH3 polypeptide, and a EPCAM polypeptide; or a CDH2 polypeptide, a LAPTM4B polypeptide, and a PODXL2 polypeptide; or a AP1M2 polypeptide, a CDH2 polypeptide, and a MARCKSL1 polypeptide; or a AP1M2 polypeptide, a CDH2 polypeptide, and a CDH3 polypeptide; or a CDH3 polypeptide, a CYP2S1 polypeptide, and a EPCAM polypeptide; or a CYP2S1 polypeptide, a HS6ST2 polypeptide, and a LMNB1 polypeptide; or a CYP2S1 polypeptide, a HS6ST2 polypeptide, and a LAPTM4B polypeptide; or a CDH2 polypeptide, a LMNB1 polypeptide, and a LRRN1 polypeptide; or a CDH2 polypeptide, a LAPTM4B polypeptide, and a SMPDL3B polypeptide; or a CYP2S1 polypeptide, a HS6ST2 polypeptide, and a KPNA2 polypeptide; or a CYP2S1 polypeptide, a HS6ST2 polypeptide, and a RCC2 polypeptide; or a GNG4 polypeptide, a LMNB1 polypeptide, and a LRRN1 polypeptide; or a CDH2 polypeptide, a LRRN1 polypeptide, and a RCC2 polypeptide; or a LAPTM4B polypeptide, a LARGE2 polypeptide, and a SMPDL3B polypeptide; or combinations thereof. In some embodiments, any two markers of the 3-biomarker combinations as described herein for detection of TGCT can be used as a 2-marker combination for detection of TGCT.

In some embodiments, at least one or more marker combinations that are suitable for detection of Thymoma (THYM) can be included in cancer detection. In some embodiments, marker combinations can enrich a population for subjects who may likely be suffering from or be susceptible to THYM.

In some embodiments, a marker combination suitable for enriching a population for subjects who may be likely suffering from or be likely susceptible to THYM comprises at least three markers, selected from the group consisting of: a CDH2 polypeptide, a CDH3 polypeptide, and a SHISA2 polypeptide; or a CDH1 polypeptide, a CDH2 polypeptide, and a HS6ST2 polypeptide; or a CDH1 polypeptide, a CDH2 polypeptide, and a SHISA2 polypeptide; or a CDH2 polypeptide, a CLN5 polypeptide, and a SHISA2 polypeptide; or a CDH2 polypeptide, a IGSF3 polypeptide, and a SHISA2 polypeptide; or a HS6ST2 polypeptide, a IGSF3 polypeptide, and a LMNB1 polypeptide; or a CDH2 polypeptide, a MARVELD2 polypeptide, and a SHISA2 polypeptide; or a CDH2 polypeptide, a LSR polypeptide, and a SHISA2 polypeptide; or a CDH1 polypeptide, a CDH2 polypeptide, and a CDH3 polypeptide; or a CDH2 polypeptide, a FERMT1 polypeptide, and a HS6ST2 polypeptide; or a HS6ST2 polypeptide, a ILDR1 polypeptide, and a SHISA2 polypeptide; or a BMPR1B polypeptide, a CDH2 polypeptide, and a HS6ST2 polypeptide; or a CDH2 polypeptide, a ILDR1 polypeptide, and a SHISA2 polypeptide; or a CDH2 polypeptide, a ILDR1 polypeptide, and a RCC2 polypeptide; or a CDH2 polypeptide, a CLN5 polypeptide, and a ILDR1 polypeptide; or combinations thereof. In some embodiments, any two markers of the 3-biomarker combinations as described herein for detection of THYM can be used as a 2-marker combination for detection of THYM.

In some embodiments, at least one or more marker combinations that are suitable for detection of Thyroid carcinoma (THCA) can be included in cancer detection. In some embodiments, marker combinations can enrich a population for subjects who may likely be suffering from or be susceptible to THCA.

In some embodiments, a marker combination suitable for enriching a population for subjects who may be likely suffering from or be likely susceptible to THCA comprises at least three markers, selected from the group consisting of: a ILDR1 polypeptide, a MET polypeptide, and a SHISA2 polypeptide; or a CDH2 polypeptide, a SHISA2 polypeptide, and a SMPDL3B polypeptide; or a CDH2 polypeptide, a CLDN3 polypeptide, and a SHISA2 polypeptide; or a CDH1 polypeptide, a CDH2 polypeptide, and a SHISA2 polypeptide; or a ILDR1 polypeptide, a SHISA2 polypeptide, and a SMPDL3B polypeptide; or a CDH2 polypeptide, a MAL2 polypeptide, and a SHISA2 polypeptide; or a CDH2 polypeptide, a EPCAM polypeptide, and a SHISA2 polypeptide; or a ILDR1 polypeptide, a MET polypeptide, and a SMPDL3B polypeptide; or a CDH2 polypeptide, a MARVELD2 polypeptide, and a SHISA2 polypeptide; or a CLN5 polypeptide, a ILDR1 polypeptide, and a SHISA2 polypeptide; or a CDH2 polypeptide, a LSR polypeptide, and a SHISA2 polypeptide; or a CDH2 polypeptide, a ILDR1 polypeptide, and a SHISA2 polypeptide; or a AP1M2 polypeptide, a CDH2 polypeptide, and a CLN5 polypeptide; or a ILDR1 polypeptide, a MET polypeptide, and a RNF128 polypeptide; or a CDH2 polypeptide, a CLN5 polypeptide, and a EPCAM polypeptide; or combinations thereof. In some embodiments, any two markers of the 3-biomarker combinations as described herein for detection of THCA can be used as a 2-marker combination for detection of THCA

In some embodiments, at least one or more marker combinations that are suitable for detection of Uterine Carcinosarcoma (UCS) can be included in cancer detection. In some embodiments, marker combinations can enrich a population for subjects who may likely be suffering from or be susceptible to UCS.

In some embodiments, a marker combination suitable for enriching a population for subjects who may be likely suffering from or be likely susceptible to UCS comprises at least three markers, selected from the group consisting of: a CDH3 polypeptide, a FZD2 polypeptide, and a SYT13 polypeptide; or a CDH2 polypeptide, a CLDN3 polypeptide, and a FZD2 polypeptide; or a CLDN3 polypeptide, a FZD2 polypeptide, and a LAPTM4B polypeptide; or a LAPTM4B polypeptide, a PODXL2 polypeptide, and a SMPDL3B polypeptide; or a CDH2 polypeptide, a LAPTM4B polypeptide, and a SMPDL3B polypeptide; or a FZD2 polypeptide, a LMNB1 polypeptide, and a VTCN1 polypeptide; or a CDH3 polypeptide, a EPCAM polypeptide, and a FZD2 polypeptide; or a CDH2 polypeptide, a LSR polypeptide, and a SHISA2 polypeptide; or a FZD2 polypeptide, a KPNA2 polypeptide, and a VTCN1 polypeptide; or a CDH2 polypeptide, a LAPTM4B polypeptide, and a PODXL2 polypeptide; or a CDH2 polypeptide, a CLDN3 polypeptide, and a LAPTM4B polypeptide; or a EPCAM polypeptide, a HS6ST2 polypeptide, and a LRRN1 polypeptide; or a FZD2 polypeptide, a SMPDL3B polypeptide, and a VTCN1 polypeptide; or a CDH3 polypeptide, a CLDN3 polypeptide, and a FZD2 polypeptide; or a APOO polypeptide, a CLDN3 polypeptide, and a FZD2 polypeptide; or combinations thereof. In some embodiments, any two markers of the 3-biomarker combinations as described herein for detection of UCS can be used as a 2-marker combination for detection of UCS.

In some embodiments, at least one or more marker combinations that are suitable for detection of Uterine Corpus Endometrial Carcinoma (UCEC) can be included in cancer detection. In some embodiments, marker combinations can enrich a population for subjects who may likely be suffering from or be susceptible to UCEC.

In some embodiments, a marker combination suitable for enriching a population for subjects who may be likely suffering from or be likely susceptible to UCEC comprises at least three markers, selected from the group consisting of: a FZD2 polypeptide, a SMPDL3B polypeptide, and a VTCN1 polypeptide; or a CLDN3 polypeptide, a LAPTM4B polypeptide, and a TMEM132A polypeptide; or a EPCAM polypeptide, a FZD2 polypeptide, and a VTCN1 polypeptide; or a BMPR1B polypeptide, a CLDN3 polypeptide, and a MARCKSL1 polypeptide; or a CLDN3 polypeptide, a FZD2 polypeptide, and a LAPTM4B polypeptide; or a BMPR1B polypeptide, a EPCAM polypeptide, and a MARCKSL1 polypeptide; or a APOO polypeptide, a CLDN3 polypeptide, and a FZD2 polypeptide; or a CLDN3 polypeptide, a FZD2 polypeptide, and a VTCN1 polypeptide; or a LAPTM4B polypeptide, a PODXL2 polypeptide, and a SMPDL3B polypeptide; or a LAPTM4B polypeptide, a SMPDL3B polypeptide, and a VTCN1 polypeptide; or a AP1M2 polypeptide, a BMPR1B polypeptide, and a SMPDL3B polypeptide; or a BMPR1B polypeptide, a CLDN3 polypeptide, and a SMPDL3B polypeptide; or a CLDN3 polypeptide, a LMNB1 polypeptide, and a VTCN1 polypeptide; or a BMPR1B polypeptide, a SERINC2 polypeptide, and a SMPDL3B polypeptide; or a FZD2 polypeptide, a PODXL2 polypeptide, and a SMIM22 polypeptide; or combinations thereof. In some embodiments, any two markers of the 3-biomarker combinations as described herein for detection of UCEC can be used as a 2-marker combination for detection of UCEC.

In some embodiments, at least one or more marker combinations that are suitable for detection of Uveal Melanoma (UVM) can be included in cancer detection. In some embodiments, marker combinations can enrich a population for subjects who may likely be suffering from or be susceptible to UVM.

In some embodiments, a marker combination suitable for enriching a population for subjects who may be likely suffering from or be likely susceptible to UVM comprises at least three markers, selected from the group consisting of: a GALNT14 polypeptide, a LAPTM4B polypeptide, and a PODXL2 polypeptide; or a LAPTM4B polypeptide, a PODXL2 polypeptide, and a SMPDL3B polypeptide; or a CDH2 polypeptide, a LAPTM4B polypeptide, and a PODXL2 polypeptide; or a CDH2 polypeptide, a GALNT14 polypeptide, and a PODXL2 polypeptide; or a LRRN1 polypeptide, a PODXL2 polypeptide, and a SLC39A6 polypeptide; or a BMPR1B polypeptide, a CDH1 polypeptide, and a PODXL2 polypeptide; or a CDH1 polypeptide, a HS6ST2 polypeptide, and a PODXL2 polypeptide; or a GJB1 polypeptide, a IGSF3 polypeptide, and a RAP2B polypeptide; or a CDH1 polypeptide, a HS6ST2 polypeptide, and a MET polypeptide; or combinations thereof. In some embodiments, any two markers of the 3-biomarker combinations as described herein for detection of UVM can be used as a 2-marker combination for detection of UVM.

What is claimed is:

1. A method for disease screening, the method comprising the steps of:
conducting a first assay for co-occurrence of two or more predetermined biomarkers on extracellular vesicles isolated from a biological sample;
conducting a second assay for indicia of disease, the target of which is different than the predetermined biomarkers; and
identifying a positive screen as a positive result in both said first assay and said second assay.

2. The method of claim 1, wherein the second assay is conducted on the same biological sample as the first assay.

3. The method of claim 1, wherein the second assay is an image-based test.

4. The method of claim 1, wherein the biological sample is tumor tissue and the second assay is a blood-based assay.

5. The method of claim 1, wherein the predetermined markers are cell-surface proteins that co-occur on extracellular vesicles released from tumor cells.

6. The method of claim 4, wherein the second assay comprises sequencing circulating tumor DNA (ctDNA).

7. The method of claim 1, wherein the two or more predetermined biomarkers are independently selected from the group consisting of CEACAM6, HS6ST2, PODXL2; LARGE2, MARCKSL1, MAL2, SMPDL3B, LSR, RAP2B, AP1M2, APOO, ALDH18A1, CDH3, NUP210, GPR160, RCC2, and LAMC2.

8. The method of claim 1, wherein the two or more predetermined biomarkers are independently selected from the group consisting of ILDR1, PODXL2, ULBP2, HS6ST2, LAMB3, LMNB1, AP1M2, CDH1, LAMC2, RAP2B, RACGAP1, LSR, CDH3, and EPCAM.

9. The method of claim 1, wherein the first assay comprises:
contacting the sample material with at least first and second oligo-linked probes;
ligating together first and second oligos of the probes to form a covalently contiguous ligation product; and
detecting the ligation product by amplification or sequencing.

10. The method of claim 9, wherein the amplification comprises digital PCR.

11. The method of claim 9, wherein, prior to the first assay, a predetermined marker comprising a surface protein of an extracellular vesicle is captured by antibody-functionalized beads, and wherein the sample material comprises extracellular vesicles.

12. The method of claim 1, wherein the sample material is immobilized on a solid substrate.

13. The method of claim 12, wherein the solid substrate is a bead.

14. The method of claim 1, wherein the sample is blood or plasma.

15. The method of claim 14, wherein the blood sample has been subjected to size exclusion chromatography to isolate extracellular vesicles.

16. The method of claim 1, wherein the predetermined markers and the disease-specific markers are specific to cancer.

17. The method of claim 16, wherein the cancer is selected from the group comprising bladder cancer, brain cancer, breast cancer, cervical cancer, chronic lymphocytic leukemia, chronic myeloid leukemia, colorectal cancer, endometrial cancer, esophageal cancer, gastrointestinal cancer, Hodgkin lymphoma, kidney cancer, liver cancer, lung cancer, multiple myeloma, non-Hodgkin lymphoma, ovarian cancer, pancreatic cancer, prostate cancer, sarcomas, skin cancer, and stomach cancer.
